SEQUENCE LISTING (3 sequences)

SEQ ID NO: 1 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 2 - protein, 102 aa, Artificial Sequence
  SLAMF7 IgG V-like domain
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHV

SEQ ID NO: 3 - protein, 76 aa, Artificial Sequence
  SLAMF7 IgG C2-like domain
PKVTMGLQSNKNGTCVTNLTCCMEHGEEDVIYTWKALGQAANESHNGSILPISWRWGESDMTFICVARNPVSRNFS

(19) United States
(12) Patent Application Publication
(10) Pub. No.: US 2019/0085334 A1
(43) Pub. Date: Mar. 21, 2019

(54) TREATMENT OF NEUROINFLAMMATION IN NEUROLOGICAL DISORDERS

(71) Applicant: Board of Trustees of Michigan State University, East Lansing, MI (US)

(72) Inventors: Yasser A. Aldhamen, East Lansing, MI (US); Patrick Thomas O'Connell, Okemos, MI (US)

(73) Assignee: Board of Trustees of Michigan State University, East Lansing, MI (US)

(21) Appl. No.: 16/131,880

(22) Filed: Sep. 14, 2018

Related U.S. Application Data
(60) Provisional application No. 62/559,027, filed on Sep. 15, 2017.

(51) Int. Cl. C12N 15/113; C12N 15/62; A61K 35/17; A61P 31/18; C12N 5/0783

(52) U.S. Cl. CPC C12N 15/1132; C12N 15/62; C12N 5/0638; A61P 31/18; A61K 35/17

(57) ABSTRACT

A method of decreasing the production of a pro-inflammatory cytokine in an activated cell of myeloid lineage in a subject in need thereof is provided. The method includes increasing at least one of signaling lymphocytic activation molecule F7 (SLAMF7) expression, SLAMF7 activity, and SLAMF7 signaling in the activated cell of myeloid lineage.

### CROSS-REFERENCE TO RELATED APPLICATIONS

This application claims the benefit of U.S. Provisional Application No. 62/559,027, filed on Sep. 15, 2017. The entire disclosure of the above application is incorporated herein by reference.

### GOVERNMENT RIGHTS

This invention was made with government support under AI122808 awarded by the National Institutes of Health. The government has certain rights in the invention.

### FIELD

The present disclosure relates to methods of activating SLAMF7 receptors and decreasing neuroinflammation in subjects in need thereof.

### BACKGROUND

This section provides background information related to the present disclosure which is not necessarily prior art.

Approximately 36.7 million people are living with human immunodeficiency virus (HIV) worldwide, with 2.1 million people becoming infected in 2015. The global market for HIV therapeutics is expected to grow to $18.2 billion in 2013 and $19.6 in 2018, with a compound annual growth rate (CAGR) of 1.5%. More than 70% of the world's HIV+ population live in Sub-Saharan Africa. In the United States, an estimated 1.1 million people are HIV+. HIV is responsible for depleting the CD4+ helper T cells within the body, which are crucial for mounting an adaptive immune response. Individuals with CD4+ helper T cell counts below 200 are diagnosed with AIDS (normal count 500-1500). Fortunately, pharmaceutical breakthroughs in antiretroviral drug development in the 1990s have allowed for HIV+ individuals to control viral load and prevent AIDS associated death.

HIV infected individuals are at risk for developing neuroinflammatory diseases such as HIV-associated neurocognitive disorder (HAND) or HIV-associated dementia (HAD), which is thought to arise due to neurological inflammation. In a cross sectional cohort study, CNS HIV AntiRetroviral Therapy Effects Research (CHARTER), researchers found that 814 (52%) of 1555 HIV-seropositive patients exhibited signs of cognitive impairment. The underlying cause of HAND and HAD remains unclear, but inflammation in the CNS has been implicated. Evidence has shown that HIV can persist in the central nervous system despite antiretroviral therapy, and animal models of HIV infection in the brain have resulted in the development of viral encephalitis and neuronal apoptosis. As shown in FIG. 1, the advent of combination antiretroviral therapy since 1996 has improved health outcomes of HIV+ individuals; however, the proportion of individuals suffering from neurological diseases have remained unchanged.

Prior to the development of combination antiretroviral therapy (cART), HIV+ diagnosis would eventually lead to the development of AIDS and individuals would be at severe danger of dying from secondary infection due to a weakened immune system. Since then, cART has made living with HIV manageable, such that individuals can live with low virus titers without risk of developing AIDS. Although the prevalence of AIDS has decreased with cART and levels of HIV kept to a minimum, approximately 50% individuals are susceptible to developing neurological symptoms which may be due to the presence of neuronal inflammation, despite having low levels of HIV. Thus, the HIV market may benefit from a technology that can reduce levels of inflammation and thereby reduce the development of neurological disease. CXCL10 is a protein produced by immune cells that acts as an “inflammatory” signal and can induce the production of several other inflammatory cytokines such as TNF-α and IL-6. Pharmaceutical companies have sought to control CXCL10 levels in an effort to reduce protein expression and thereby decrease levels of inflammation using monoclonal antibodies, which have been well tolerated and safe. However, the anti-inflammatory properties have been shown to be limited in phase II clinical trials. There remains a need to develop methods for controlling or decreasing the levels of inflammatory cytokines in individuals with, for example, HIV.

### SUMMARY

This section provides a general summary of the disclosure, and is not a comprehensive disclosure of its full scope or all of its features.

In various aspects, the current technology provides a method of decreasing the production of a pro-inflammatory cytokine in an over activated cell of myeloid lineage of a subject in need thereof. The method includes increasing at least one of signaling lymphocytic activation molecule F7 (SLAMF7) expression, SLAMF7 activity, and SLAMF7 signaling in the activated cell of myeloid lineage.

In one aspect, the pro-inflammatory cytokine is selected from the group consisting of interleukin-1β (IL-1p), interleukin-6 (IL-6), interleukin-17a (IL-17a), tumor necrosis factor α (TNFα), a chemokine, and combinations thereof.

In one aspect, the chemokine is C-X-C motif chemokine 9 (CXCL9), C-X-C motif chemokine 10 (CXCL10), C-X-C motif chemokine 11 (CXCL11), C-X-C motif chemokine 12 (CXCL12), or a combination thereof.

In one aspect, the subject in need thereof has a disorder selected from the group consisting of HIV-associated neurocognitive disorder (HAND), HIV-associated dementia (HAD), breast cancer, atherosclerosis, coronary artery disease, sepsis, systemic lupus erythematosus, myocardial infarction, Alzheimer's disease, Huntington's disease, multiple sclerosis, obesity, kidney disease, Rheumatoid arthritis, and a combination thereof.

In one aspect, the increasing at least one of SLAMF7 expression, SLAMF7 activity, and SLAMF7 signaling includes administering a safe and effective amount of nuclear factor-κβ (NF-κβ), palmitoyl cysteine serine lysine 4 (pamCSK4), histone deacetylase 1 activator (HDAC1), histone deacetylase 2 activator (HDAC2), p300 inhibitor, a CD3 activator, a CD28 activator, a DNA molecule capable of expressing a SLAMF7 gene or gene fraction, a component that decreases YY1 levels, a component that prevents or decreases acetylation of YY1, interferon α (IFNα), interferon β (IFNβ), interferon γ (IFNγ), lipopolysaccharide (LPS), Adenovirus, polyinosinic:polycytidylic acid (poly I:C), flagelin, guanosine-adenosine 2′,3′-cyclic monophosphate (2′3′-cGAMP), resiquimod (R848), SLAMF7 agonistic antibodies, SLAMF7 agonistic antibody fractions, a hybrid molecule comprising a portion of SLAMF7 and an antibody Fc fragment, a small molecule agonist of SLAMF7, a micelle or liposome having recombinant SLAMF7 on a surface, or a combination thereof to the subject.

In one aspect, the DNA molecule is a plasmid or a minicircle.

In one aspect, the administering the DNA molecule includes transfecting cells of the myeloid lineage by a virus, electroporation, direct microinjection, laser-mediated transfection, cationic lipid transfection, squeezing the cell to create an opening in the cell's membrane, or calcium phosphate transfection.

In one aspect, the cell of myeloid lineage is derived from a multipotential hematopoietic stem cell.

In one aspect, cell of myeloid lineage is a monocyte, a neutrophil, or a microglia.

In one aspect, the cell of myeloid lineage is derived from an erythro-myeloid precursor in an embryonic yolk sac.

In one aspect, the cell of myeloid lineage is a microglia.

In various aspects, the current technology also provides a method of decreasing the production of a pro-inflammatory cytokine in an activated cell of myeloid lineage of a subject in need thereof. The method includes administering a safe and effective amount of a signaling lymphocytic activation molecule F7 (SLAMF7) agonist to the subject. The SLAMF7 agonist increases at least one of SLAMF7 expression, SLAMF7 activity, and SLAMF7 signaling in the activated cell of myeloid lineage, wherein the activated cell of myeloid lineage is a monocyte, neutrophil, or microglia that produces pro-inflammatory cytokines at a level that is higher than baseline.

In one aspect, the subject has HIV-associated neurocognitive disorder (HAND) or HIV-associated dementia (HAD).

In one aspect, the method further includes treating the subject with combination antiretroviral therapy (cART).

In one aspect, the SLAMF7 agonist is a SLAMF7 agonistic antibody, a SLAMF7 agonistic antibody fragment, a hybrid molecule comprising a portion of SLAMF7 and an antibody Fc fragment, a small molecule, or an agent that increases SLAMF7 expression and signaling in the activated cell of myeloid lineage.

In various aspects, the current technology yet further provides a method of inhibiting human immunodeficiency virus (HIV) from infecting a cell of either myeloid lineage or lymphoid lineage. The method includes modulating the cell to at least one of decrease an amount of chemokine receptor type 5 (CCR5) in a cell membrane of the cell or increase an amount of C-C motif chemokine ligand 3 like 1 (CCL3L1) expressed in the cell.

In one aspect, the modulating is performed by increasing at least one of signaling lymphocytic activation molecule F7 (SLAMF7) expression, SLAMF7 activity, and SLAMF7 signaling in the cell.

In one aspect, the increasing at least one of SLAMF7 expression, SLAMF7 activity, and SLAMF7 signaling in the cell is performed by contacting the cell with palmitoyl cysteine serine lysine 4 (pamCSK4), histone deacetylase 1 activator (HDAC1), histone deacetylase 2 activator (HDAC2), a p300 inhibitor, a CD3 activator, a CD28 activator, a DNA molecule capable of expressing a SLAMF7 gene, a component that decreases YY1 levels, a component that prevents or decreases acetylation of YY1, interferon α (IFNα), interferon β (IFNβ), interferon γ (IFNγ), lipopolysaccharide (LPS), Adenovirus, polyinosinic:polycytidylic acid (poly I:C), flagelin, guanosine-adenosine 2′,3′-cyclic monophosphate (2′3′-cGAMP), resiquimod (R848), SLAMF7 agonistic antibodies, SLAMF7 agonistic antibody fractions, a small molecule agonist of SLAMF7, a peptide, a micelle or liposome having recombinant SLAMF7 on a surface, or a combination thereof.

In one aspect, the cell is a monocyte or a T cell.

In one aspect, the cell is in a subject having human immunodeficiency virus (HIV) or in a subject at risk of having HIV.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 shows pie charts adapted from the prior art. The charts show that proportions of neurocognitive disease remain similar among HIV+ patients since the introduction of combination antiretroviral therapy (CART). In the charts, HAD refers to HIV-associated dementia, ANI refers to asymptomatic neurocognitive impairment, and MND refers to mild neurocognitive disorder.

FIG. 2 is a schematic illustration showing an overview of a SLAMF7 activation pathway. HIV+ patient classical (CD14+CD16−) monocytes produce CXCL10 with type I IFN stimulation. The production of CXCL10 induces inflammation in the central nervous system. Activation of SLAMF7 inhibits the production of CXCL10 of classical monocytes and also results in reduction of inflammatory mediators such as IL-6 and TNF-α.

FIG. 3 is an illustration showing the activation of SLAMF7 with and without SLAMF7 association with EAT-2.

FIG. 4 is an illustration showing that SLAMF7 activation on monocytes is able to decrease their susceptibility to HIV-1 infection in vitro via down-regulation of CCR5 and up-regulation of the CCL3L1 chemokine.

FIG. 5 shows a monocyte and neutrophil gating strategy.

FIGS. 6A-6K show HIV+ individuals have increased levels of SLAMF7 on their PBMCs and SLAMF7 is up-regulated by IFNα. HIV+ patients and healthy controls (HC) were screened for total SLAMF7 expression across all peripheral immune cell types (FIG. 6A). SLAMF7 expression is assessed on CD4 T cells (FIG. 6B), CD8 T cells (FIG. 6C), and NKT cells (FIG. 6D). The n is indicated along the x-axis (FIG. 6A) and the white diamonds indicate the mean. The n for FIG. 6B and FIG. 6C is: HC: 10, Concordant: 28, Discordant: 5, LTNP: 3, EC: 4. The n for FIG. 6D is: HC: 14, HIV: 26. The effect of IFNα (100 IU/mL) on SLAMF7 expression on total peripheral blood mononuclear cells (PBMCs) (FIG. 6E and FIG. 6F), and monocytes (FIG. 6J and FIG. 6K) was assessed in vitro. For FIG. 6F and FIG. 6K, n=46 and 26, respectively and data are pooled from 5 independent experiments. FIG. 6G and FIG. 6H show CD14 expression on SLAMF7^{high} cells from IFNα stimulated total PBMCs. FIG. 6I is a comparison of SLAMF7 expression on CD14⁺ and CD14⁻ cells following IFNα stimulation, showing only CD14⁺ monocytes up-regulate SLAMF7. Groups were compared using 1-way ANOVA with Tukey's multiple comparison test (FIG. 6A) and paired two-tailed T test (FIG. 6C and FIG. 6E). *P<0.05, ****P<0.0001. LTNP refers to long-term non-progressor and ns refers to not significant.

FIG. 7 is a bar graph showing that removing DNA methylation does not affect SLAMF7 expression.

FIG. 8 is a dot plot showing SLAMF7 levels in monocytes and neutrophils after histone deacetylase treatment.

FIG. 9 is a dot plot showing SLAMF7 levels in classical monocytes after histone deacetylase treatment and treatment with IFNα and IFNα plus the histone deacetylase inhibitor (HDACi) Dacinostat (LAQ824). The plot shows that HDACi treatment prevents IFNα-mediated SLAMF7 up-regulation.

FIG. 10 is a dot plot showing that following SLAMF7 down-regulation, HDACi treatment up-regulates CXCL10 expression monocytes and neutrophils.

FIGS. 11A-11C show that SLAMF7 down-regulation may occur via increased levels of acetylated YY1. Addition of a pan HDAC inhibitor or an HDAC 1/2 specific inhibitor robustly downregulates SLAMF7 expression on monocytes (FIG. 11A). Neutrophils contain higher levels of YY1 (FIG. 11B). FIG. 11C is an illustration showing a proposed mechanism explaining acetylation dependent regulation of SLAMF7 by YY1.

FIGS. 12A-12B show transcription factor Klf4 levels in neutrophils. FIGS. 12A and 12B show that neutrophils have a lower level of Klf4 than classical monocytes.

FIGS. 13A-13G show activation of the SLAMF7 receptor on monocytes inhibits their IFNα-mediated production of CXCL10. In FIGS. 13A and 13B, PBMCs from healthy controls were stimulated in vitro with IFNα and the SLAMF7 receptor was activated by cross-linking where indicated. Expression of CXCL10 was measured by intracellular staining on flow cytometry. In FIGS. 13C and 13D, the same experiment in FIG. 13A was carried out with isolated CD14⁺ monocytes. Cross-linking with a recombinant protein comprised of the extracellular domain of SLAMF7 fused to a modified IgG4 Fc fragment (SLAMF7-Fc) was performed to confirm that inhibition is SLAMF7 specific (FIGS. 13C and 13D, last condition). In FIG. 13E, the levels of secreted CXCL10 in the supernatant from FIG. 13A was assessed by BioPlex assay. In FIGS. 13F and 13G, PBMCs from HIV+ donors were isolated and the same experiment as in FIG. 13A was carried out. HIV patients failing to respond to SLAMF7 activation were classified as SLAMF7 silent (SF7S). In FIGS. 13B and 13E, n=7. In FIG. 13D, n=4 technical replicates from a single donor, representative of 3 independent experiments. In FIGS. 13B and 13E, data are presented as pooled results of 2 independent experiments, representative of 7 total experiments. In FIGS. 13F and 13G, data presented are pooled from 2 independent experiments. 3-4 HCs were run alongside HIV samples in all experiments to verify that assay worked. SF7S and SLAMF7 responsive groups are compared using 2-way ANOVA with Sidak's multiple comparison test. Data in FIGS. 13B, 13D, and 13E are presented as mean±SEM. Groups are compared using 1-way ANOVA with Tukey's multiple comparison test. *P<0.05, **P<0.01, ***P<0.001, ****P<0.0001.

FIGS. 14A-14E show SLAMF7 activation inhibits IFNγ-mediated CXCL10 production from monocytes and CXCL10 inhibition is not proteasome-mediated. FIG. 14A shows CXCL10⁺ levels in classical monocytes contacted with IFNγ. FIG. 14B shows CXCL10⁺ levels in classical monocytes contacted with IFNα. FIG. 14C shows SLAMF7 levels in monocytes after being contacted with IFNα. FIG. 14D shows the result of flow cytometry experiments. FIG. 14E is a dot plot showing CXCL1⁺ levels in classical monocytes after being contacted with IFNα.

FIGS. 15A-15C show SLAMF7 silent patients have elevated plasma levels of proinflammatory factors and cluster distinctly from SLAMF7 responsive patients. Plasma from HIV patients and HCs was assessed by BioPlex assay for 6 proinflammatory cytokines and chemokines known to be involved in HIV-associated immune dysfunction (CXCL10, MIP-1β, IL-6, IL-8, G-CSF and MCP-1). In FIG. 15A, k-means clustering was performed with all 6 factors and plots depicting all relationships are shown. Previous hierarchal clustering identified two distinct clusters (data not shown). Dots indicate cluster 1 (n=28) and cluster 2 (n=12). FIG. 15B shows heatmap of z-score normalized plasma cytokine and chemokine values. FIG. 15C shows a breakdown of percentages of SF7S and SLAMF7 responsive patients per cluster. The number of patients per group, per cluster are indicated inside bars.

FIGS. 16A-16D show monocytes from SF7S and SLAMF7 responsive patients do not differ in expression levels of SLAMF7 or EAT-2. FIG. 16A shows expression of SLAMF7 on monocyte subsets. FIG. 16B shows a comparison of SLAMF7 expression between SF7S, SLAMF7 responsive, and HCs on all monocyte subsets. In FIG. 16C, levels of EAT-2, a known SLAM family receptor adaptor, are assessed in a human cell line known to express EAT-2 (NK92) and isolated primary monocytes by qRT-PCR. FIG. 16D shows an analysis of EAT-2 protein levels in monocytes between SF7S and SLAMF7 responsive individuals by intracellular staining on flow cytometry. In FIG. 16C, results are technical replicates from a single donor compared with unpaired 2-tailed T test. Groups in FIGS. 16B and 16D are compared using 1-way ANOVA with Tukey's multiple comparison test, as well as a 2-way ANOVA with Sidak's multiple comparison. In FIG. 16B, n=6 for HCs, 8 for SLAMF7 responsive, and 5 for SF7S. In FIG. 16D, n=3 for HCs, 2 for SLAMF7 responsive, and 5 for SF7S. Data are presented as mean±SEM. ***P<0.001.

FIGS. 17A-17D show SLAMF7 activation on monocytes is selective for alpha chemokines and may not be mediated by any of the inhibitory phosphatases known to interact with SLAMF7. In FIGS. 17A and 17B, the effects of SLAMF7-mediated inhibition of other alpha chemokines (FIG. 17A) and ISGs (FIG. 17B) are assessed in isolated monocytes from HCs at the mRNA level by qRT-PCR. In FIG. 17C, the effects of SHP1/2 and SHIP1 on SLAMF7-mediated inhibition of CXCL10 was assessed via small molecule inhibitors (SHP1/2: NSC87877, 10 μM) (SHIP1: 3AC, 5 μM). SHP1/2 effects on alpha chemokines were also assessed at the mRNA level (FIG. 17A). In FIG. 17D, the role of CD45 in SLAMF7-mediated inhibition of CXCL10 was assessed via a small molecule inhibitor (CAS 345630-40-2, 1 μM). FIGS. 17A and 17B are representative of 4 independent experiments. Groups in FIGS. 17A-17D were compared using 1-way ANOVA with Tukey's multiple comparison test. Data are presented as mean±SEM. *P<0.05, **P<0.01, ***P<0.001, ****P<0.0001.

FIGS. 18A-18E show SLAMF7 activation inhibits monocyte infection with HIV-1 in vitro and down-regulates CD16. In FIGS. 18A and 18B, PBMCs from HIV+ individuals (n=6) were stimulated in vitro with IFNα and the SLAMF7 receptor was activated by cross-linking where indicated. Surface expression of CCR5 was measured by flow cytometry. In FIG. 18C, mRNA expression of CCL3L1 was assessed by qRT-PCR from the same samples in FIGS. 17A and 17B. In FIG. 18D, isolated monocytes from 2 HCs (labeled “A” and “B”) were infected with HIV-1_{-Ba-L}-GFP and infectivity was assessed at the indicated time points by FACS. In FIG. 18E, PBMCs from a single HC were stimulated in vitro as indicated for 24 hours before analysis by flow cytometry. This experiment used 25 IU/mL IFNα. Percent indicated on y-axis is from all cells in FSC-A/SSC-A “monocyte” gate. In FIGS. 18B and 18C, groups are compared using a 1-way ANOVA with Tukey's multiple comparison test. In FIG. 18E, groups are compared using a 2-way ANOVA with Tukey's multiple comparison test. Data are presented as mean±SEM and is representative of 1 experiment (FIGS. 18B, 18C, and 18D) or 2 independent experiments (FIG. 18E). *P<0.05, **P<0.01, ***P<0.001, ****P<0.0001.

FIGS. 19A-19F show neutrophils are SLAMF7- and CXCL10⁺ and do not respond to SLAMF7 activation. Baseline SLAMF7 and CXCL10 expression was examined on CD14⁺CD16⁻ monocytes and neutrophils by flow cytometry (FIG. 19A). FIG. 19B shows the quantification of results from FIG. 19A, focusing on CXCL10⁺ cells. FIG. 19C shows neutrophils do not increase SLAMF7 expression in response to IFNα stimulation. In FIGS. 19D-19F, isolated neutrophils from HCs and HIV patients were stimulated in vitro with IFNα, LPS, and SLAMF7 as indicated and supernatants were assessed by BioPlex assay for indicated cytokines/chemokines. In FIG. 19B, n=30. In FIG. 19C, n=3 independent donors from a single experiment. In FIGS. 19D-19F, the data presented are pooled from 2 independent experiments with n=10 donors. In FIG. 19B, groups are compared using 2-way ANOVA with Sidak's multiple comparison test. Groups in FIG. 19C are compared using 2-tailed T test. Groups in FIGS. 19D-19F are compared using 1-way ANOVA with Tukey's multiple comparison test. Data are presented as mean±SEM. *P<0.05, ****P<0.0001.

FIGS. 20A-20B are bar charts showing that SLAMF7 activation prevents type I interferon-mediated production of IL-6 (FIG. 20A) and TNFα (FIG. 20B) from human microglia.

FIGS. 21A-21B are bar charts showing that SLAMF7 activation prevents IFNγ (FIG. 21A) and TNFα (FIG. 21B) production from human microglia cells following CXCL10 stimulation.

FIG. 22 is a bar chart showing that SLAMF7 activation inhibits IL-6 production from Aβ stimulated human microglia cells (HMC3).

FIGS. 23A-23D are bar charts showing that SLAMF7 activation inhibits CD80 and CD86 co-stimulatory receptor expression from Aβ stimulated human microglia cells (HMC3). FIGS. 23A and 23B show CD80 levels in microglia cells after Aβ and IFNγ stimulation. FIGS. 23C and 23D show CD86 levels in microglia cells after Aβ and IFNγ stimulation.

FIGS. 24A-24B show that SLAMF7 activation in human microglia induces production of microglia-derived neuroprotective factor FGF-basic. FIG. 24A shows levels of FGF-basic in microglia after contact with SLAMF7 cross-linking and FIG. 24B shows levels of FGF-basic in microglia after contact with various agents.

FIG. 25 is a bar graph showing that SLAMF7 activation in human microglia cells enhances production of the neuronal survival cytokine IL-7.

FIGS. 26A-26B are bar graphs showing that SLAMF7 activation in human microglia cells induces production of the anti-inflammatory cytokine IL-9. FIG. 26A shows levels of IL-9 in microglia after contact with SLAMF7 cross-linking and FIG. 26B shows levels of IL-9 in microglia after contact with various agents.

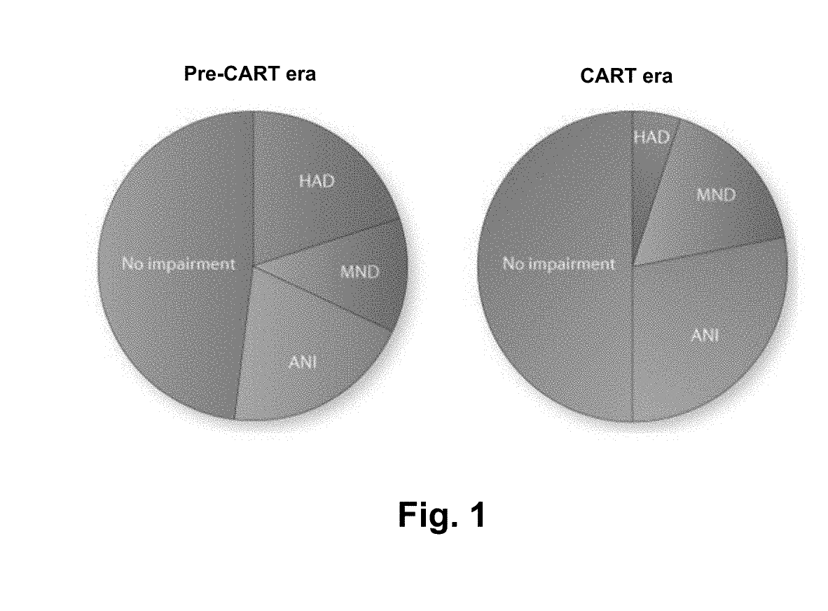

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges. As referred to herein, ranges are, unless specified otherwise, inclusive of endpoints and include disclosure of all distinct values and further divided ranges within the entire range. Thus, for example, a range of “from A to B” or “from about A to about B” is inclusive of A and B.

Example embodiments will now be described more fully with reference to the accompanying drawings.

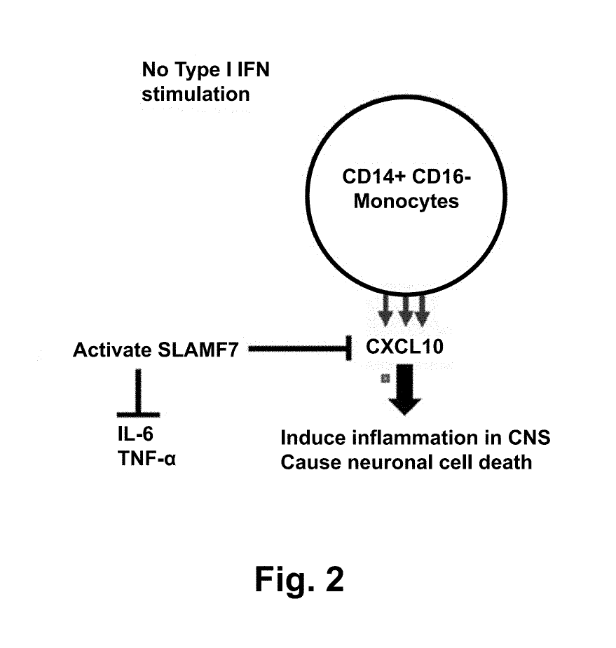

The current technology provides a mechanism to suppress the production of pro-inflammatory cytokines through the activation of an immune receptor present on innate neuronal immune cells of HIV+ individuals. More specifically, and with reference to FIG. 2, the current technology provides the activation of SLAMF7/CD319/CS-1, a self-associating CRACC protein which negatively regulates the expression of CXCL10, an inflammatory chemokine that stimulates monocytes (which can differentiate into macrophages) and promotes natural killer cell and T-cell migration. The current technology provides for the activation of SLAMF7 by overexpression and or crosslinking of SLAMF7 dimers, which will result in a decrease in production of other inflammatory cytokines, such as TNF-α and IL-6. Other approaches to reduce inflammatory CXCL10 have utilized monoclonal antibodies (BMS-936557-Brisol-Myers Squibb/NI-0801-Novimmune), but have been found to have limited anti-inflammatory activity. Additionally, this technology offers an improved approach to reducing inflammation by preventing, decreasing, or inhibiting the secretion of broad variety inflammatory chemokines rather than targeting one class. By suppressing the production of inflammatory signals, this technology increases therapeutic potential in treatment of HIV-associated neurological disease.

The current technology provides the benefits of reducing neuroinflammation for HIV patients and providing a novel pathway for immune modulation. Moreover, the current technology can be applied to preventing, decreasing, suppressing, or inhibiting HIV, HIV-induced neurological disease, HIV-associated neurocognitive disorder (HAND), and HIV-associated dementia (HAD) or for treating type I diabetes, Alzheimer's Disease, and Huntington's Disease, as non-limiting examples.

Diseases with increased non-classical monocytes and diseases that can be treated by the current technology include: HIV associated neurocognitive disorder, HIV associated dementia, breast cancer, atherosclerosis (“Recent work attempting to differentiate the contribution of CD16+ monocytes to atherosclerosis show that intermediate monocyte frequency is closely related to severity of angina and may contribute to atherosclerosis”), coronary artery disease, sepsis, systemic lupus erythematosus, myocardial infarction (“peak levels of CD14++CD16− monocytes were a negative predictor of myocardial salvage and no relationship between CD16+ monocytes and infarction size was noted in this study”), Alzheimer disease, multiple sclerosis, obesity (“Several groups have independently demonstrated that the frequency of non-classical and intermediate monocytes positively correlates with WHO obesity classification and fat mass”), kidney disease (“Patient cohorts have uniformly demonstrated that increased intermediate (CD14++CD16+) monocyte frequency is associated with cardiovascular event rate and increased mortality. A study of 119 patients with CKD demonstrated an increased frequency of intermediate monocytes in the peripheral blood of hemodialysis-dependent CKD patients compared to CKD patients with adequate native renal function. Depletion of intermediate monocytes during hemodialysis was associated with an increased cardiovascular event-free period and reduced mortality in patients with end-stage renal disease. While CD14++CD16+ monocytes appear to be an important biomarker of CKD severity, their role in the evolution of CKD is yet to be determined.”), and Rheumatoid arthritis (“CD16+ monocytes are found in the synovial fluid of RA patients and are associated with joint destruction. Elevation of the CD16+ monocyte frequency in RA patients was recently demonstrated to be primarily an expansion of the intermediate population. Sequestering the inflammatory properties of CD14++CD16+ monocytes may inhibit joint injury and signify response to therapy.”).

Signaling from the SLAMF7 receptor can inhibit CXCL 10 production from type I interferon-stimulated primary human CD14+CD16− monocytes of HIV patients and healthy controls. Additionally, neuroinflammation-inducing neutrophils from HIV+ individuals are CRACC negative and consequently produce large amounts of CXCL 10 upon stimulation by IFNα or IFNγ. Because the neutrophils are CRACC negative, the CXCL10 cannot be inhibited by activating SLAMF7. High levels of CXCL 10 in the central nervous system (CNS) can induce neuroinflammation and cause neuronal cell death in multiple neurological disorders (such as Alzheimer's Disease, Multiple Sclerosis, Huntington Disease) and infectious diseases associated with neuroinflammation. In HIV patients this leads to a phenomenon known as a HIV-associated neurocognitive disorder (HAND) or HIV-associated dementia (HAD). Additionally, CXCL 10 can activate the brain-resident immune and non-immune cells, such as microglia and astrocytes, causing them to produce proinflammatory cytokines (such as IL-6 and TNFa), which can further induce neuroinflammation and neuronal apoptosis.

Investigating the role of SLAMF7 in a human microglia cell line (HMC3) reveals that SLAMF7 activation completely inhibited the production of the proinflammatory cytokines IL-6 and TNFa from these cells, both at the baseline and following type I interferon, LPS, poly(I:C), and recombinant CXCL-10 protein stimulation. Surprisingly, it was found that SLAMF7 activation in microglia cells induced a significant amount of the neuroprotective growth factors FGF-basic, the Glial cell-derived neurotropic factor (GDNF), and the neuronal survival cytokine, IL-7. These responses were also associated with increased levels of the anti-inflammatory cytokine IL-9, a cytokine that is known to promote neuroprotective responses and neuronal survival.

Current results show that the SLAMF7 receptor is a critical negative regulator of both CXCL10, IL-6, and TNFα in myeloid-lineage cells and that neutrophils in HIV-infected patients can down-regulate SLAMF7 specifically to allow them to remain constitutively active, thus inducing HAND and HAD. Therefore, modulating SLAMF7 receptor signaling in these cells is a therapeutic strategy to prevent neuroinflammation and enhance neuronal cell survival.

The current technology provides a mechanism that suppresses the production of pro-inflammatory cytokines and chemokines in the CNS by activating the SLAMF7 receptor signaling or inducing SLAMF7 expression in cells of the myeloid lineage, including microglia and the CD14^{low} CD16+non-classical monocytes.

Methods explored to carry this out include, but are not limited to: use of SLAMF7-activating small molecules, peptides, natural compounds, agonistic antibodies or antibody conjugates, alteration of SLAMF7 gene expression, alteration of chromatin modifications and alteration of cell metabolism.

Factors that up-regulate SLAMF7, by activation or expression, include, as non-limiting examples, nuclear factor-κβ (NF-κβ), palmitoyl cysteine serine lysine 4 (pamCSK4), histone deacetylase 1 activator (HDAC1), histone deacetylase 2 activator (HDAC2), p300 activator, a CD3 activator, a CD28 activator, a DNA molecule capable of expressing a SLAMF7 gene or gene fraction, interferon α (IFNα), interferon β (IFNβ), interferon γ (IFNγ), lipopolysaccharide (LPS), Ad, polyinosinic:polycytidylic acid (poly I:C), flagelin, guanosine-adenosine 2′,3′-cyclic monophosphate (2′3′-cGAMP), resiquimod (R848), SLAMF7 agonistic antibodies, SLAMF7 agonistic antibody fractions, a small molecule agonist of SLAMF7, a peptide (such as a SLAMF7 peptide), a micelle or liposome having recombinant SLAMF7 on a surface, or combinations thereof. Table 1 shows amino acid sequence for SLAMF7 and of various peptides that are useful, individually or collectively, for activating SLAMF7.

TABLE 1
Amino acid sequences.

| | SEQ | |
|---|---|---|
| | ID | |
| Molecule | NO: | Sequence |
| SLAMF7 | 1 | MAGSPTCLTLIYILWQLTGSAASGPVKELVGS |
| | | VGGAVTFPLKSKVKQVDSIVWTFNTTPLVTIQ |
| | | PEGGTIIVTQNRNRERVDFPDGGYSLKLSKLK |
| | | KNDSGIYYVGIYSSSLQQPSTQEYVLHVYEHL |
| | | SKPKVTMGLQSNKNGTCVTNLTCCMEHGEEDV |
| | | IYTWKALGQAANESHNGSILPISWRWGESDMT |
| | | FICVARNPVSRNFSSPILARKLCEGAADDPDS |
| | | SMVLLCLLLVPLLLSLFVLGLFLWFLKRERQE |
| | | EYIEEKKRVDICRETPNICPHSGENTEYDTIP |
| | | HTNRTILKEDPANTVYSTVEIPKKMENPHSLL |
| | | TMPDTPRLFAYENVI |
| SLAMF7 | 2 | SGPVKELVGSVGGAVTFPLKSKVKQVDSIVWT |
| IgG | | FNTTPLVTIQPEGGTIIVTQNRNRERVDFPDG |
| V-like | | GYSLKLSKLKKNDSGIYYVGIYSSSLQQPSTQ |
| domain | | EYVLHV |
| SLAMF7 | 3 | PKVTMGLQSNKNGTCVTNLTCCMEHGEEDVIY |
| IgG | | TWKALGQAANESHNGSILPISWRWGESDMTFI |
| C2-like | | CVARNPVSRNFS |
| domain | | |

Among the SLAMF7 agonists describe herein, the peptides of SEQ ID NO:1 and SEQ ID NO:2, and peptides that are greater than or equal to about 80%, greater than or equal to about 85%, greater than or equal to about 90%, greater than or equal to about 95% identical to the peptides of SEQ ID NO:1 and SEQ ID NO:2, individually and collectively, are agonists of SLAMF7.

Accordingly, the current technology provides a method of decreasing the production of a pro-inflammatory cytokine in an activated cell of myeloid lineage in a subject in need thereof. As used herein, an “activated” cell is a cell that produces pro-inflammatory cytokines at a level that is higher than a baseline level for the cell. The cell of myeloid lineage is derived from a multipotential hematopoietic stem cell or from an erythro-myeloid precursor in an embryonic yolk sac. The cell of myeloid lineage derived from a multipotential hematopoietic stem cell is a monocyte, a neutrophil, a microglia, or a combination thereof. The monocyte can be a classical monocyte (CD14⁺⁺CD16⁻), a non-classical monocyte (CD14⁺CD16⁺⁺), an intermediate monocyte (CD14⁺⁺CD16⁺), an inflammatory monocyte, or a combination thereof. The cell of myeloid lineage derived from an erythro-myeloid precursor in an embryonic yolk sac is a microglia.

The pro-inflammatory cytokines are interleukin-1β (IL-1p), interleukin-6 (IL-6), interleukin-17a (IL-17a), tumor necrosis factor α (TNFα), a chemokine, or combinations thereof. Exemplary chemokines include C-X-C motif chemokine 9 (CXCL9), C-X-C motif chemokine 10 (CXCL10), C-X-C motif chemokine 11 (CXCL11), C-X-C motif chemokine 12 (CXCL12), and combinations thereof.

The subject in need of decreasing the production of the pro-inflammatory cytokine in an over-activated cell of myeloid lineage has a disorder that is associated with an over-activated cells of myeloid lineage. The disorder is selected from the group consisting of HIV-associated neurocognitive disorder (HAND), HIV-associated dementia (HAD), breast cancer, atherosclerosis, coronary artery disease, sepsis, systemic lupus erythematosus, myocardial infarction, Alzheimer's disease, Huntington's disease, multiple sclerosis, obesity, kidney disease, Rheumatoid arthritis, and a combination thereof.

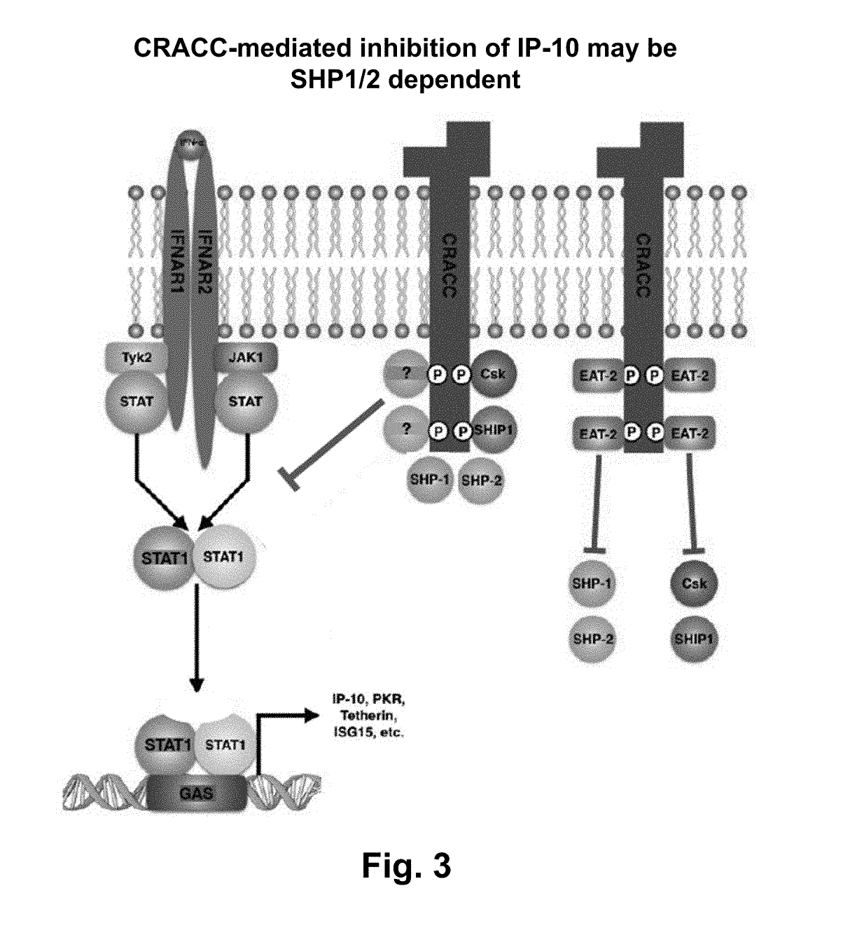

The method comprises increasing at least one of SLAMF7 expression, SLAMF7 activity, and SLAM7 signaling in the over-activated cell of myeloid lineage. In various embodiments, the SLAMF7 in the over-activated cell is not associated with any, or a sufficient amount of, Ewing's sarcoma-associated transcript 2 (EAT-2) SLAM-associated protein adapter (SAP), such that the increasing the at least one of SLAMF7 expression, SLAMF7 activity, and SLAM7 signaling results in the activation of inhibitory phosphatases or other factors that lead to downstream inhibition of the pro-inflammatory cytokines. As shown in FIG. 3, the activation of the inhibitory phosphatases results in a decrease in the production of the pro-inflammatory cytokine in the over-activated cell of myeloid lineage.

In some embodiments, the increasing at least one of SLAMF7 expression, SLAMF7 activity, and SLAMF7 signaling in the over-activated cell of myeloid lineage comprises administering a safe and effective amount of a SLAMF7 agonist to the subject. The SLAMF7 agonist is nuclear factor-κβ (NF-κβ), palmitoyl cysteine serine lysine 4 (pamCSK4), histone deacetylase 1 activator (HDAC1), histone deacetylase 2 activator (HDAC2), p300 inhibitor, a CD3 activator, a CD28 activator, a DNA molecule capable of expressing a SLAMF7 gene or gene fraction, a component that decreases YY1 levels, a component that prevents or decreases acetylation of YY1, interferon α (IFNα), interferon β (IFNβ), interferon γ (IFNγ), lipopolysaccharide (LPS), Adenovirus, polyinosinic:polycytidylic acid (poly I:C), flagelin, guanosine-adenosine 2′,3′-cyclic monophosphate (2′3′-cGAMP), resiquimod (R848), SLAMF7 agonistic antibodies, SLAMF7 agonistic antibody fractions, a hybrid molecule comprising a portion of SLAMF7 and an antibody Fc fragment, a small molecule agonist of SLAMF7, a peptide (such as a SLAMF7 peptide), a micelle or liposome having recombinant SLAMF7 on a surface, or a combination thereof. Administering the DNA molecule to the subject comprises transfecting cells of the myeloid lineage by a virus, electroporation, direct microinjection, laser-mediated transfection, cationic lipid transfection, squeezing the cell to create an opening (or plurality of openings) in the cell's membrane, or calcium phosphate transfection. Various SLAMF7 agonists are described in U.S. patent application Ser. No. 15/513,660, filed on Mar. 23, 2017 by Amalfitano et al., published as U.S. Publication No. 2017/0292127 on Oct. 12, 2017, which is incorporated herein by reference in its entirety.

In other embodiments, the increasing at least one of SLAMF7 expression, SLAMF7 activity, and SLAMF7 signaling in the over-activated cell of myeloid lineage comprises isolating the cell of myeloid lineage from the subject, and contacting the cell with a SLAMF7 agonist. The increasing the at least one of SLAMF7 expression, SLAMF7 activity, and SLAMF7 signaling in the over-activated cell of myeloid lineage causes the cell of myeloid lineage to decrease production of pro-inflammatory cytokines. Subsequently, the cell of myeloid lineage is transfused back into the subject.

In various embodiments, the subject has HIV-associated neurocognitive disorder (HAND) or HIV-associated dementia (HAD) and the method further comprises treating the subject with combination antiretroviral therapy (cART).

The current technology also provides a method of inhibiting human immunodeficiency virus (HIV) from infecting a cell of either myeloid lineage or lymphoid lineage. As described above, the cell of myeloid lineage is derived from a multipotential hematopoietic stem cell or from an erythro-myeloid precursor in an embryonic yolk sac. The cell of myeloid lineage derived from a multipotential hematopoietic stem cell is a monocyte, a neutrophil, a microglia, or a combination thereof. The monocyte can be a classical monocyte (CD14⁺⁺CD16⁻), a non-classical monocyte (CD14⁺CD16⁺⁺), an intermediate monocyte (CD14⁺⁺CD16⁺), an inflammatory monocyte, or a combination thereof. The cell of myeloid lineage derived from an erythro-myeloid precursor in an embryonic yolk sac is a microglia. The cell of lymphoid lineage is also derived from a multipotential hematopoietic stem cell. The cell of lymphoid lineage derived from a multipotential hematopoietic stem cell is a T lymphocyte, i.e., a T cell, and more specifically, can be a CD4⁺ T cell.

The method comprises modulating the cell to at least one of decrease an amount of chemokine receptor type 5 (CCR5) in a cell membrane of the cell or increase an amount of C-C motif chemokine ligand 3 like 1 (CCL3L1) expressed in the cell. The modulating is performed by increasing at least one of signaling lymphocytic activation molecule F7 (SLAMF7) expression, SLAMF7 activity, and SLAMF7 signaling in the cell.

The increasing at least one of SLAMF7 expression and SLAMF7 activity in the cell is performed by contacting the cell with a SLAMF7 agonist. The SLAMF7 agonist can be any SLAMF7 agonist or combination of SLAMF7 agonists described herein.

In some embodiments, the cell is in a subject having HIV and the cell is a monocyte. Accordingly, performing the method minimizes, decreases, inhibits, or prevents the subject from developing a HAND, such as HAD. Here, the method is performed either by administering the SLAMF7 agonist to the subject or by isolating monocytes from the subject, contacting the monocytes with the SLAMF7 agonist, and transfusing the monocytes back into the subject.

In other embodiments, the subject is a subject at risk of being infected by HIV and the cell is a CD4⁺ T cell. Accordingly, performing the method minimizes, decreases, inhibits, or prevents the subject from acquiring HIV. Here, the method is performed by administering the SLAMF7 agonist to the subject.

Embodiments of the present technology are further illustrated through the following non-limiting examples.

### Example 1

SLAMF7 is a Critical Negative Regulator of Interferon-α-Mediated CXCL 10 Production in Chronic HIV Infection.

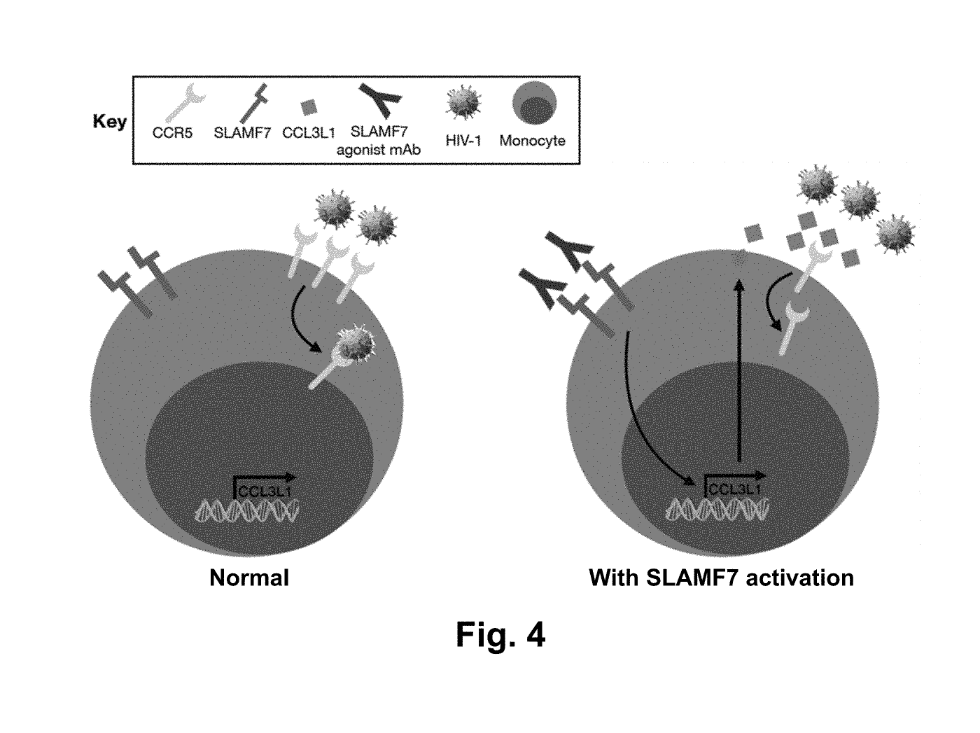

Current advances in combined anti-retroviral therapy (cART) has rendered HIV infection a chronic, manageable disease; however, the problem of persistent immune activation still remains, despite treatment. The immune cell receptor SLAMF7 has been shown to be upregulated in diseases characterized by chronic immune activation. Here, the function of the SLAMF7 receptor in immune cells of HIV patients and the impacts of SLAMF7 signaling on peripheral immune activation is studied. Increased frequencies of SLAMF7⁺ PBMCs in HIV+ individuals is observed in a clinical phenotype-dependent manner, with discordant and long-term nonprogressor patients showing elevated SLAMF7 levels, and elite controllers showing levels comparable to healthy controls. It was also found that SLAMF7 is sensitive to IFNα stimulation; a factor elevated during HIV infection. Further studies reveal SLAMF7 to be a potent inhibitor of the monocyte-derived proinflammatory chemokine CXCL10 (IP-10) and other CXCR3 ligands, except in a subset of HIV+ patients termed SLAMF7 silent (SF7S). Studies utilizing small molecule inhibitors reveal that the mechanism of CXCL10 inhibition is independent of known SLAMF7 binding partners. Furthermore, it is determined that SLAMF7 activation on monocytes is able to decrease their susceptibility to HIV-1 infection in vitro via down-regulation of CCR5 and up-regulation of the CCL3L1 chemokine as shown in FIG. 4. Finally, it is discovered that neutrophils do not express SLAMF7, are CXCL10⁺ at baseline, are able to secrete CXCL10 in response to IFNα and LPS, and are non-responsive to SLAMF7 signaling. These findings implicate the SLAMF7 receptor as an important regulator of IFNα-driven innate immune responses during HIV infection.

Introduction.

HIV infection is now widely regarded as a manageable disease; however, cART, which controls viremia, fails to effectively control many secondary HIV-associated pathologies. The universal mechanism that is believed to underlie the development of these diseases is chronic, global immune activation. Known causes of which include increased gut permeability resulting in microbial translocation into systemic circulation and constitutively elevated levels of proinflammatory cytokines and chemokines, including interferon alpha (IFNα).

Sustained levels of detectable IFNα in the plasma of cART-treated HIV patients results from persistent activation of plasmacytoid dendritic cells (pDCs) by HIV-antibody complexes and causes a global type I interferon signature in circulating monocytes. This induces monocyte transition from the classical subtype (CD14⁺CD16⁻) into the proinflammatory (CD14⁺CD16⁺) and non-classical (CD14^{low}CD16⁺⁺) subtypes, as shown by upregulation of CD16. While beneficial in acute infections, these type I interferon-mediated effects on monocytes prove to be deleterious in chronic infections.

Some HIV+ individuals are known to have persistently elevated levels of CXCL10 (IP-10) and other proinflammatory cytokines and chemokines. CXCL10 is produced primarily by monocytes and is of particular interest due to its ability to suppress T cell functions, induce neuronal apoptosis, and act as a marker of systemic inflammation. During HIV infection, some CD16⁺ monocytes become activated by IFNα, upregulate CCR5, are infected with HIV, and subsequently migrate across the blood-brain barrier where they set up a viral reservoir and are capable of inducing neuroinflammation and neuronal apoptosis via secretion of high levels of CXCL10, TNFα, IL-6, and IL-1β. Here, the role of an immune-modulatory receptor, SLAMF7 (CRACC, CS-1, CD319), is investigated in the context of HIV infection and immune activation.

SLAMF7 is a member of the signaling lymphocytic activation molecules (SLAM) family of receptors and is expressed on numerous immune cell types. SLAMF7, and other SLAM family receptors (except 2B4), function as homotypic receptors that, upon activation, recruit SLAM-associated protein (SAP) family of adaptors or other SH2 domain-containing proteins to their cytoplasmic immunoreceptor tyrosine-based switch motifs (ITSMs). SLAMF7 is unique among SLAM receptors in that it is only able to recruit a single SAP adaptor, EAT-2, to its tyrosine-phosphorylated ITSM. SLAMF7 receptor ligation in cells expressing EAT-2 results in activation of cellular immune responses, while SLAMF7 activation in the absence of EAT-2 causes recruitment of a number of inhibitory phosphatases (SHP1, SHP2, SHIP1, and csk).

SLAMF7 is well known for being over-expressed on multiple myeloma (MM) cells, as an important regulator of NK cell function, and recently, as a critical factor in macrophage-mediated phagocytosis of tumor cells. Using a medium-sized cohort of middle-aged, HIV+ individuals, it is studied whether the SLAMF7 receptor is playing a role in the context of HIV infection in cART-treated patients. Results implicate the SLAMF7 receptor as an important immunomodulatory receptor in the context of HIV-associated peripheral immune activation.

Materials and Methods.

Reagents Used.

The following antibodies are used: CD14-FITC (61D3), CD16-BV510 (3G8), SLAMF7-PE (162), CD3-PE-Cy7 (OKT3), CD19-PE-Cy7 (SJ25C1), CD57-PE-Cy7 (TB01), CD66b-APC (G10F5), CXCL10-PerCp-eFluor710 (4NY8UN), EAT-2-APC (LS-C240730), YY1-Alexa647 (H-10), Blimp-1-DyLight650 (3H2-E8), CCR5-PerCp-eFluor710 (NP-6G4) and SLAMF7 (162.1) (used for cross-linking). The SLAMF7-Fc recombinant protein is designed and produced similar to mCRACC-Fc as previously described, with the following modifications: a human SLAMF7 extracellular domain is swapped for the murine SLAMF7 extracellular domain, the murine IgG Fc portion is switched to a human IgG4 domain to reduce Fc receptor binding and ADCC, and S228P and L235E mutations are made in the IgG4 domain to further reduce interactions with Fc receptors. Recombinant universal IFNα (PBL Assay Bioscience) and Recombinant human IFNγ (ProSpec) are used at 100 IU/mL unless otherwise noted. SHP1/2 inhibitor (NSC87877) (Millipore Sigma) is used at 10 μM. SHIP1 inhibitor (3AC) (Millipore Sigma) is used at 5 μM. CD45 inhibitor (CAS 345630-40-2) (Millipore Sigma) is used at 1 μM. Bortezomib (EMD Millipore) is used at 100 nM. All HDACi's (Selleck chemicals) are used at concentrations indicated in relevant figure legends.

HC and HIV Blood Sample Collection.

HC PBMCs are obtained from either buffy coats purchased from Gulf Coast Regional Blood Center, Texas, or from whole blood samples purchased from Stanford Blood Center, California. HIV+ blood samples are collected from donors enrolled in the Mid-Michigan HIV Consortium. Plasma is stored at −80° C. until use. PBMCs are isolated with Ficoll-Plaque Plus (GE Healthcare) as previously described.

In Vitro Cell Culture and Stimuli.

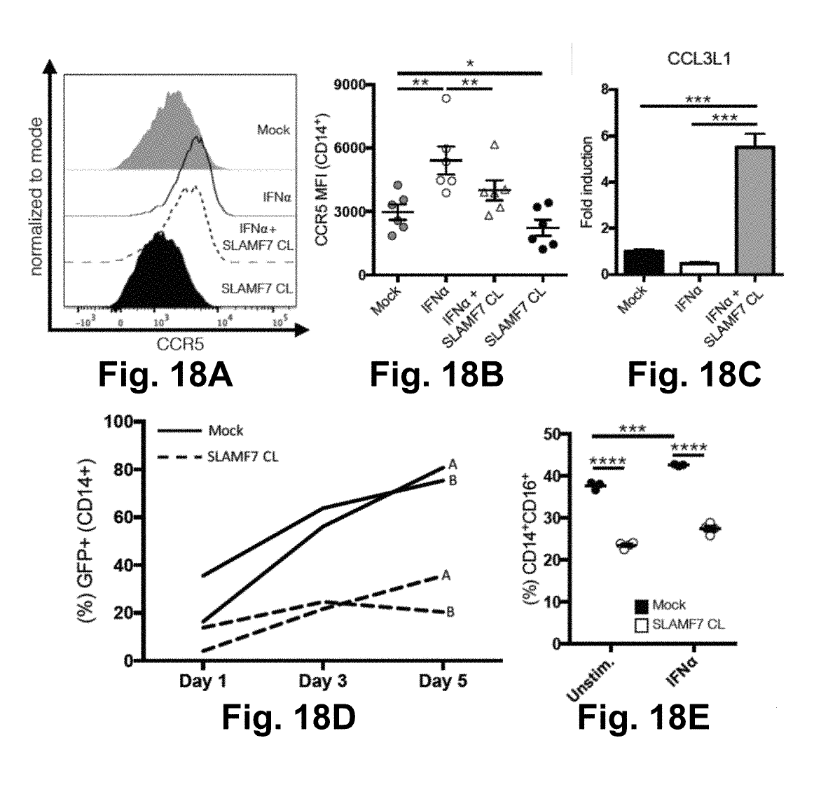

Cells are plated at 3×10⁵ cells/well for PBMC and isolated monocyte experiments, and 1×10⁵ cell/well for isolated neutrophil experiments, in 96-well plates. Cells are cultured in complete RPMI (RPMI 1640, 10% FBS, 1×PSF). For cross-linking experiments 10 μg/mL anti-SLAMF7 mAb or SLAMF7-Fc is added to a sterile, high-binding 96-well cell culture plate O/N at 4° C. For all in vitro flow cytometry experiments, cells are cultured in the presence of stimuli for 17-18 h. In the monocyte maturation assay as shown in FIG. 18E the cells are stimulated with only 25 IU/mL IFNα for exactly 24 h. Small molecule inhibitors are added 45-60 min before IFNα for all relevant experiments. For experiments involving intracellular staining, BD GolgiPlug is added for the final 5 h. NK92 cells are purchased from ATCC and cultured as indicated by manufacturer.

BioPlex Assay.

For analysis of cell culture, supernatant and plasma samples 80 μL of media or plasma is used, undiluted, and the assay is run per manufacturer's instructions (Bio-Rad, Hercules, Calif.) via Luminex 100 technology. A 27-plex assay is run and analysis is performed on only those factors with detectable levels or contributing to the separation of Cluster 1 and Cluster 2.

Flow Cytometry.

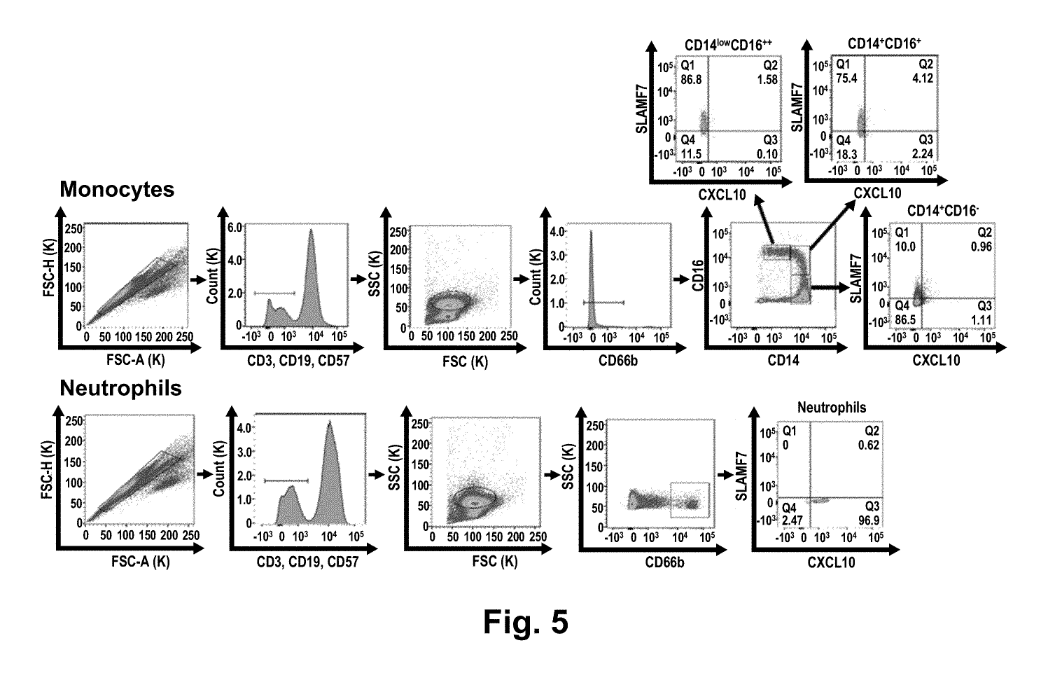

Cells are prepared and stained as previously described. Intracellular staining is performed using the BD fixation/permeabilization kit (BD Biosciences) per manufacturer's instructions. Transcription factor staining is performed with the Transcription Factor Buffer Set (BD Pharmingen) per manufacturer's instructions. Samples are analyzed on either a BD LSR II or BD FACSCanto. LIVE/DEAD staining with Aqua fixable stain (ThermoFisher) is included during initial experiments and cell viability is verified to be greater than 90%. Monocyte and neutrophil gating strategy is shown in FIG. 5.

Monocyte and Neutrophil Isolations.

CD14⁺ monocytes are isolated from PBMCs via positive selection using CD14 Microbeads (Miltenyi Biotec) per manufacturer's instructions. Purity is consistently greater than 90% as assessed by flow cytometry. Neutrophils are isolated from whole blood using the MACSxpress Neutrophil isolation kit (Miltenyi Biotec) per manufacturer's instructions. Purity is consistently greater than 98% as assessed by flow cytometry.

qRT-PCR Experiments.

Isolated monocytes are plated in 96-well plates at 3×10⁵ cells/well and stimulated as indicated for 4 h. Cells are harvested and placed into Trizol (ThermoFisher) and RNA is isolated per manufacturer's instructions. RNA is reverse transcribed with SuperStrand First Strand Synthesis Kit III (Invitrogen) per manufacturer's instructions and analyzed on a QuantStudio7 system (ThermoFisher). GAPDH is used as a housekeeping gene and the ΔΔCₜ method is used for analysis.

In Vitro HIV-1 Infection of Monocytes.

Isolated primary human monocytes from two HC's are plated at 1×10⁵ cells/well in a 96-well cell culture plate. SLAMF7 activation is induced by antibody cross-linking, the same as in IFNα stimulation experiments. Cells are cultured for 24 h before HIV-1_{Ba-L}-GFP is added at a final concentration of 190 pg/mL. Cells are washed 24 h following infection and collected at indicated time points post-infection for assessment of infectivity via FACS analysis.

Clustering and Statistical Analysis.

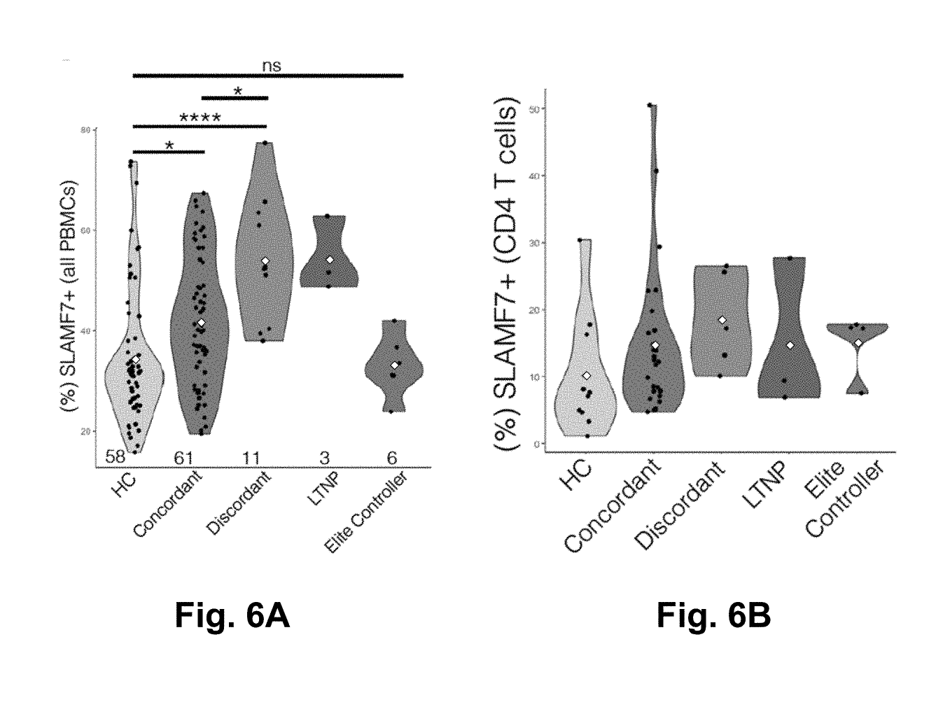

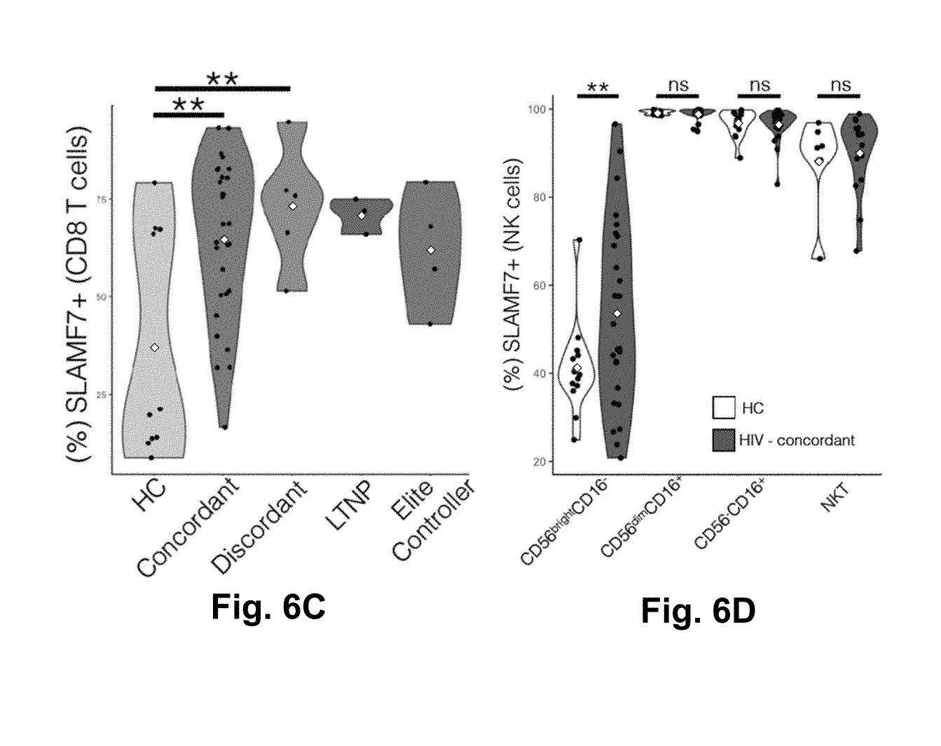

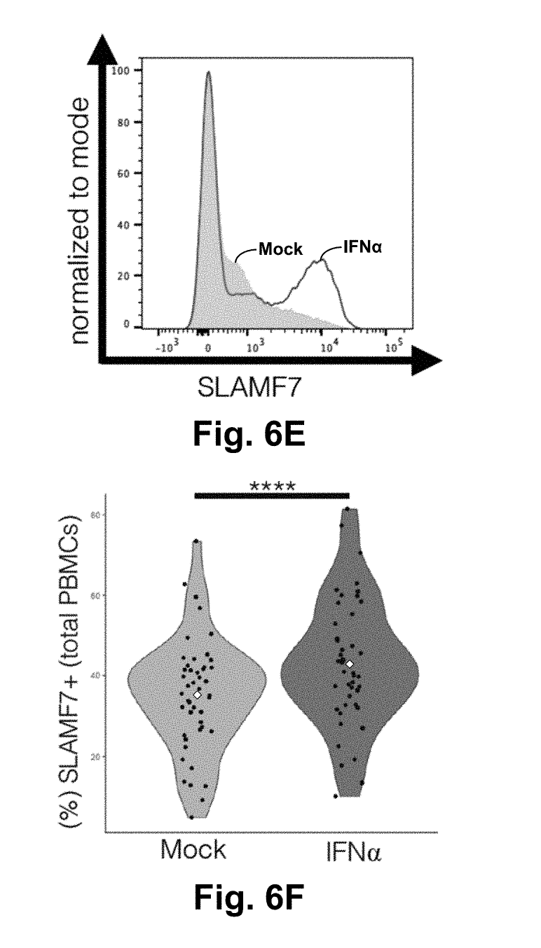

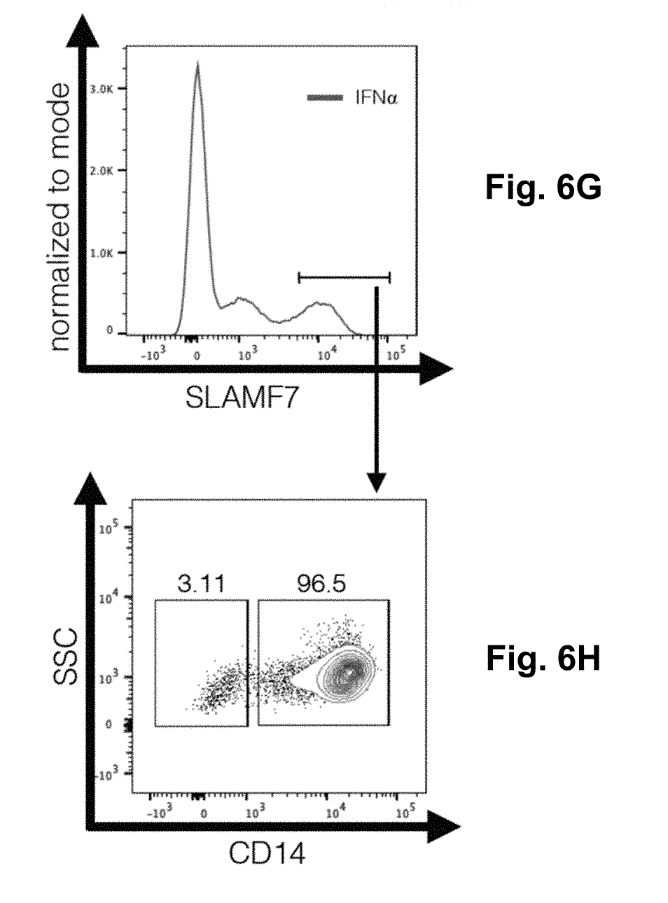

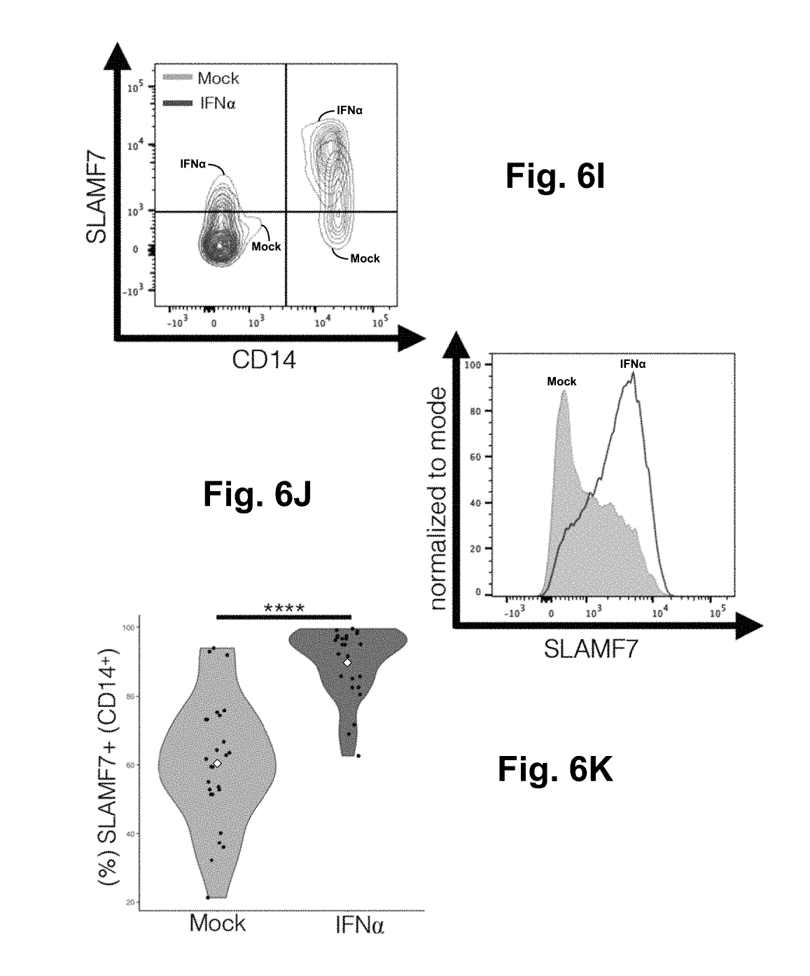

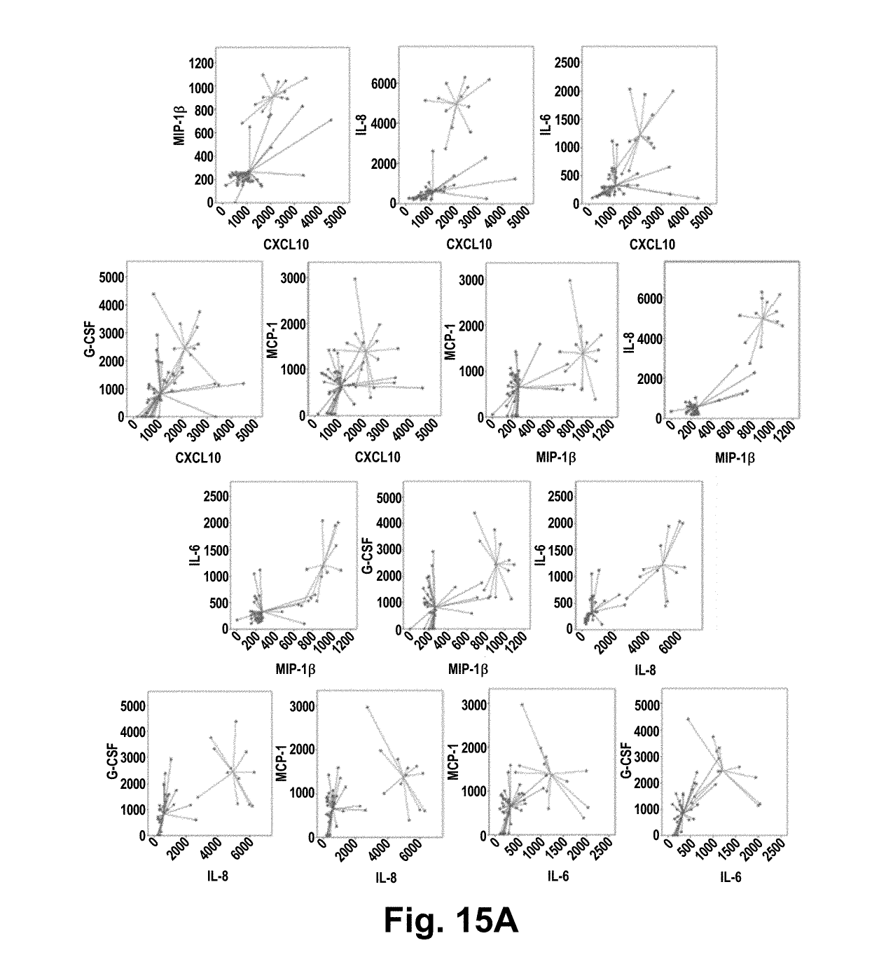

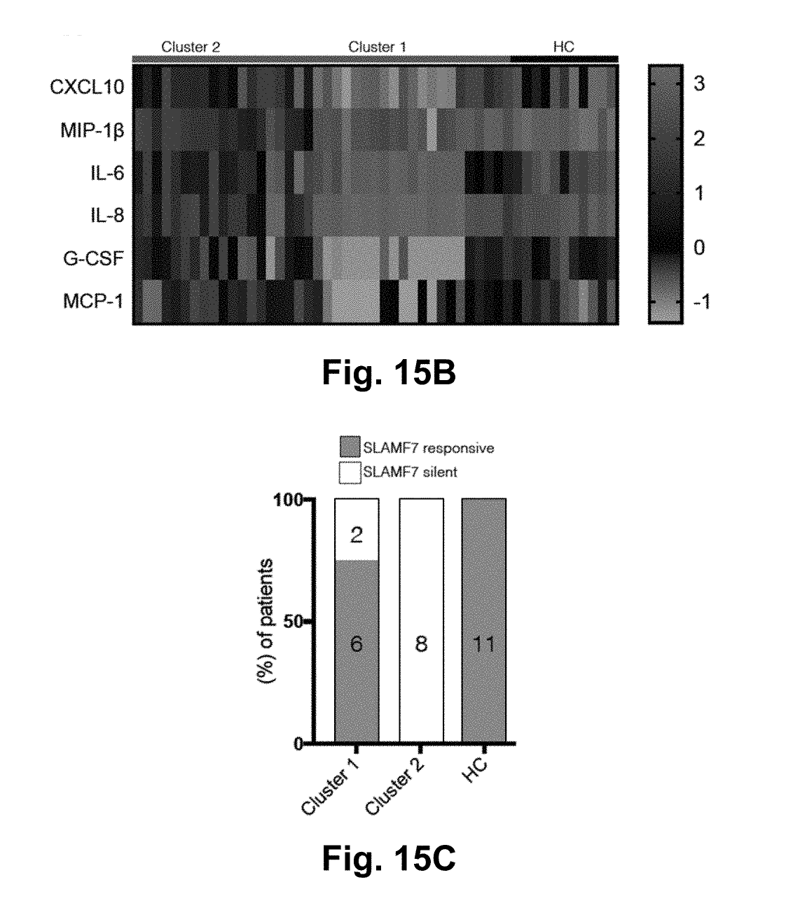

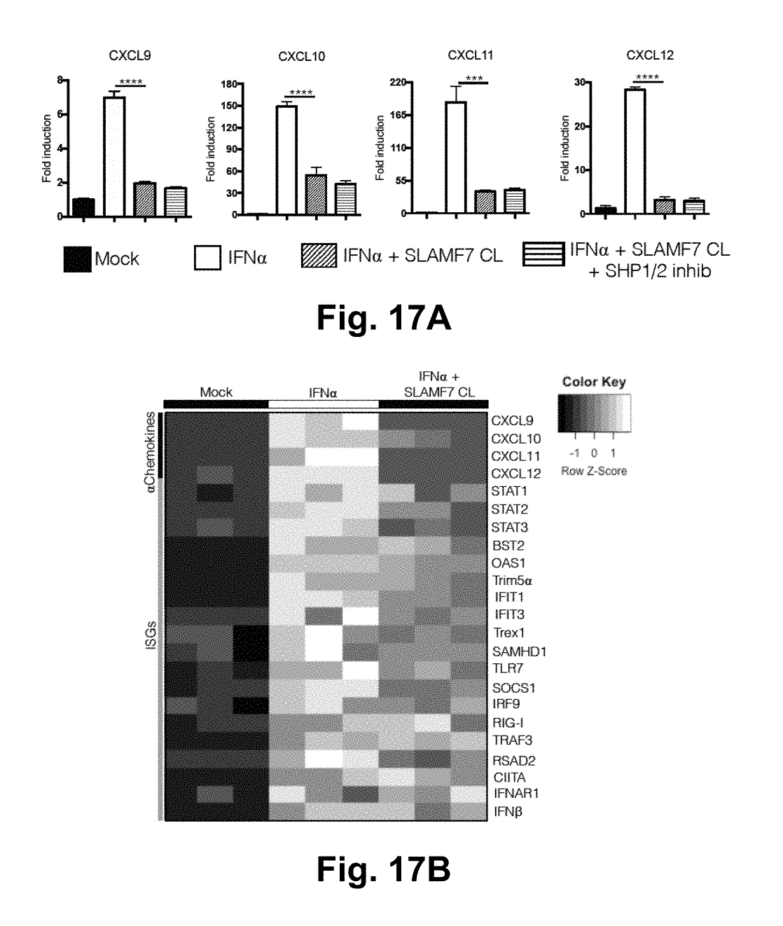

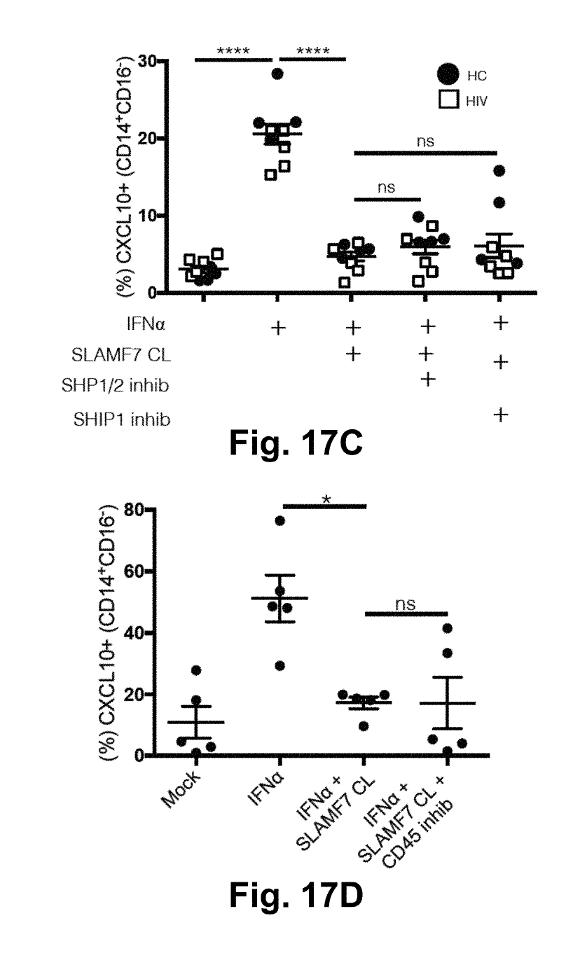

k-means and hierarchical clustering are performed using SPSS (IBM). Statistical analysis is performed in GraphPad Prism 7.0, as indicated. The heatmap from FIG. 15B is generated using the z-score normalized values of plasma biomarkers calculated in SPSS. The violin plots in FIGS. 6A-6K are generated using the ggplot2 package in R. The gene expression heatmap from FIG. 17B is generated using the gplot package in R. MFI refers to “median fluorescence intensity.”

Study Approval.

Informed consent from all HIV patients is obtained prior to their enrollment in the study and approved by the MSU IRB (IRB#: 11-202). Patient data is de-identified and complies with all HIPAA regulations.

Results.

HIV+ Individuals have Increased Expression of SLAMF7 on their Peripheral Blood Mononuclear Cells (PBMCs).

The SLAMF7 receptor functions as a self-ligand; therefore, global receptor levels across the complete spectrum of PBMCs will impact the function of all SLAMF7⁺ cells. To investigate if HIV infection alters SLAMF7 expression 81 HIV patients and 58 healthy controls (HCs) are screened for global expression of SLAMF7 across all peripheral immune cells as shown in FIG. 6A. Subjects are grouped according to their clinical phenotype: cART concordant (CD4 count >250 with no decrease in CD4 count over 6 months), cART discordant (CD4 count <250 with undetectable viral load and no improvement in CD4 count over 6 months), long-term nonprogressor (LTNP), or elite controller (EC). It is observed that SLAMF7 levels minimally increase in concordant patients, while discordant and LTNP patients have significantly higher SLAMF7 levels as shown in FIG. 6A. Interestingly, elite controllers have global SLAMF7 levels comparable to HCs as shown in FIG. 6A. Assessment of SLAMF7 expression on CD4 and CD8 T cells reveal SLAMF7 to be significantly up-regulated on CD8 T cells from concordant and discordant patients as shown in FIGS. 6B and 6C. Expression of SLAMF7 on 4 subsets of NK cells (CD56^{bright}CD16⁻, CD56^{dim}CD16⁺, CD56⁻CD16⁺, and NKT cells) show comparable expression between HCs and HIV patients as shown in FIG. 6D. Interestingly, HIV patients have significantly more SLAMF7 cells in the CD56^{bright}CD16⁻ subset, as compared to other NK cell subsets as shown in 6D.

DNA Methylation does not Cause Alterations in Cell Surface Expression of SLAMF7.

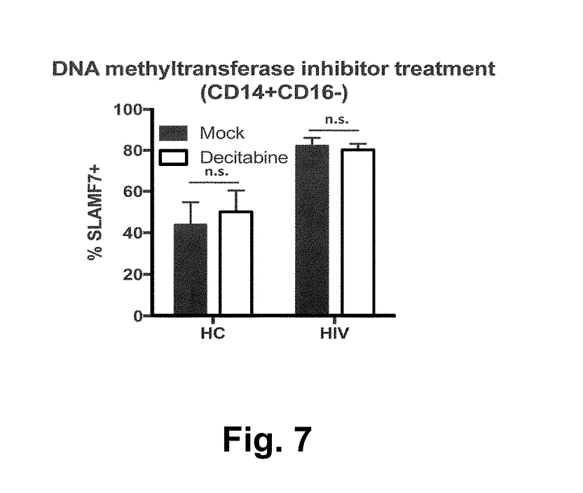

With reference to FIG. 7, removing DNA methylation does not affect SLAMF7 expression.

Histone Deacetylase (HDAC) Effect on SLAMF7 Activity.

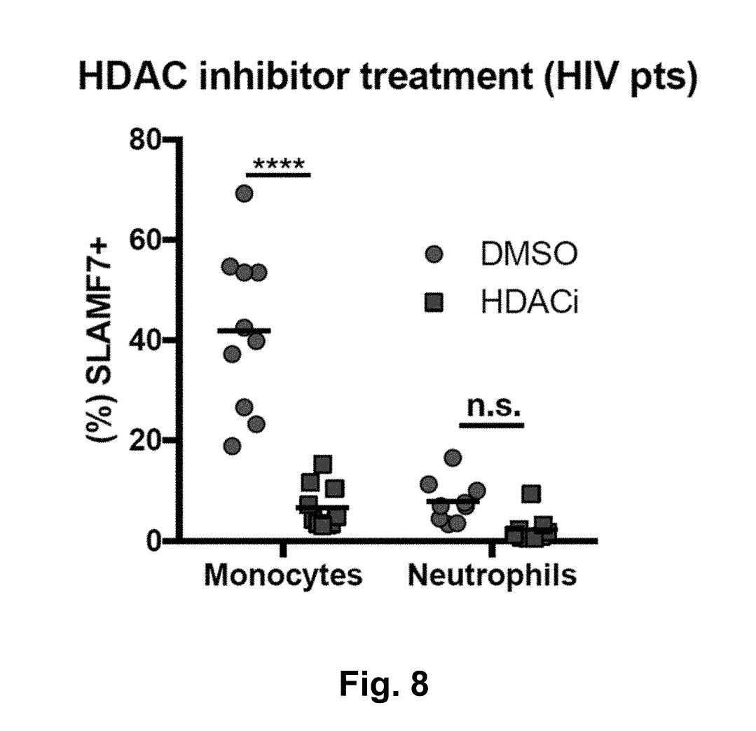

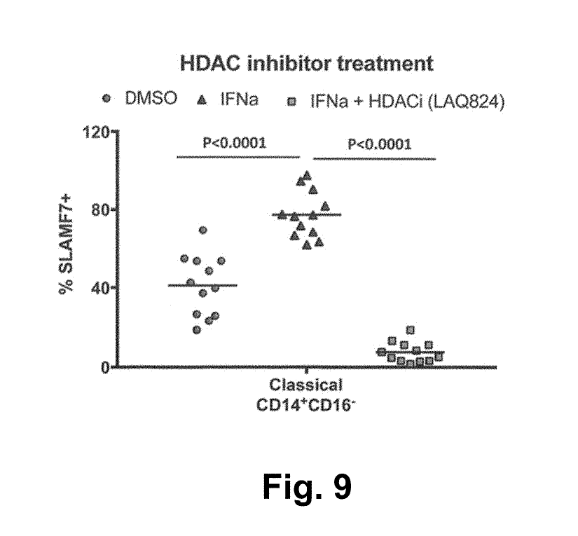

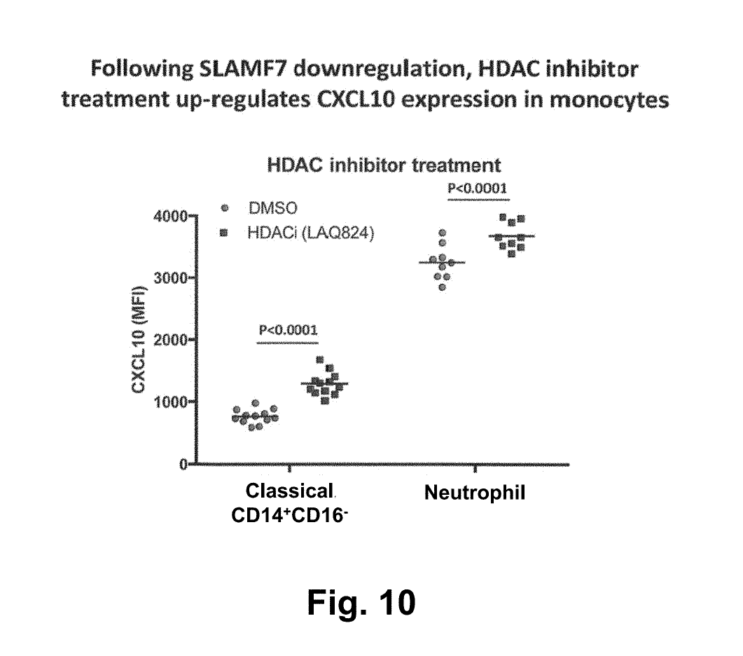

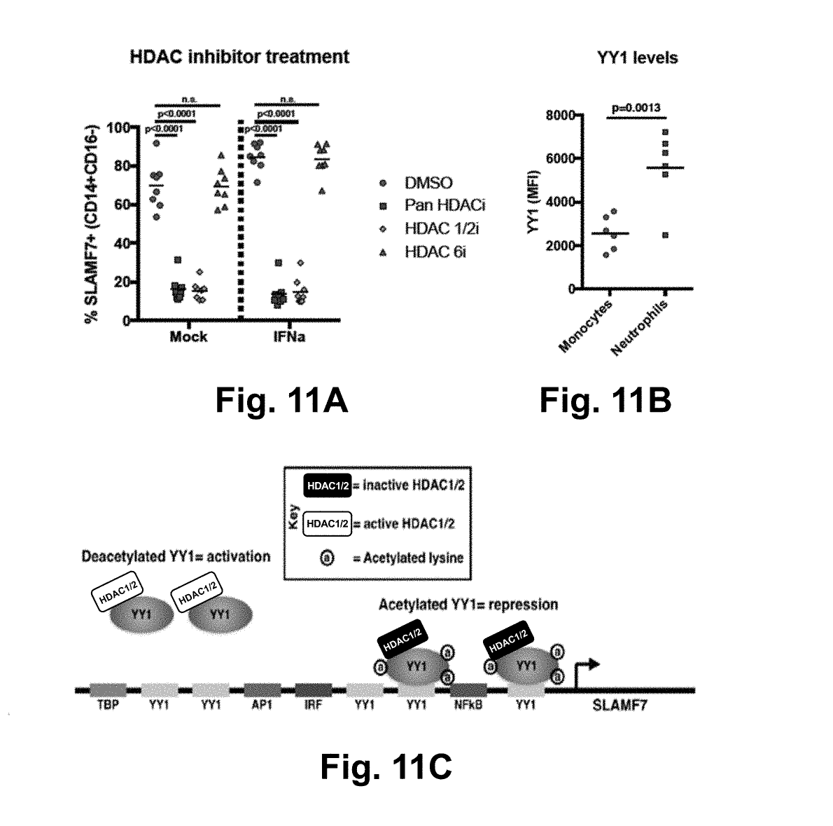

FIG. 8 shows the effect that HDAC inhibitors have on SLAMF7 levels in monocytes and neutrophils. Here it is shown that whereas SLAMF7 levels are low in neutrophils with or without HDAC inhibitors, in monocytes, the level of SLAMF7 decreases upon the addition of HDAC inhibitors. As shown in FIG. 9, HDAC inhibitor treatment prevents IFNα-mediated SLAMF7 up-regulation. With reference to FIG. 10, following SLAMF7 downregulation, HDAC inhibitor treatment up-regulates CXCL10 expression in monocytes and neutrophils. FIG. 11A shows the effect of HAC inhibitor treatment on SLAMF7 levels in cells treated with IFNα and FIG. 11B shows YY1 levels in monocytes and neutrophils. FIG. 11C is a cartoon showing possible relationships between YY1 and HDAC1/2 in regard to SLAMF7 transcription.

Transcription Factor KIf4 Levels in Neutrophils.

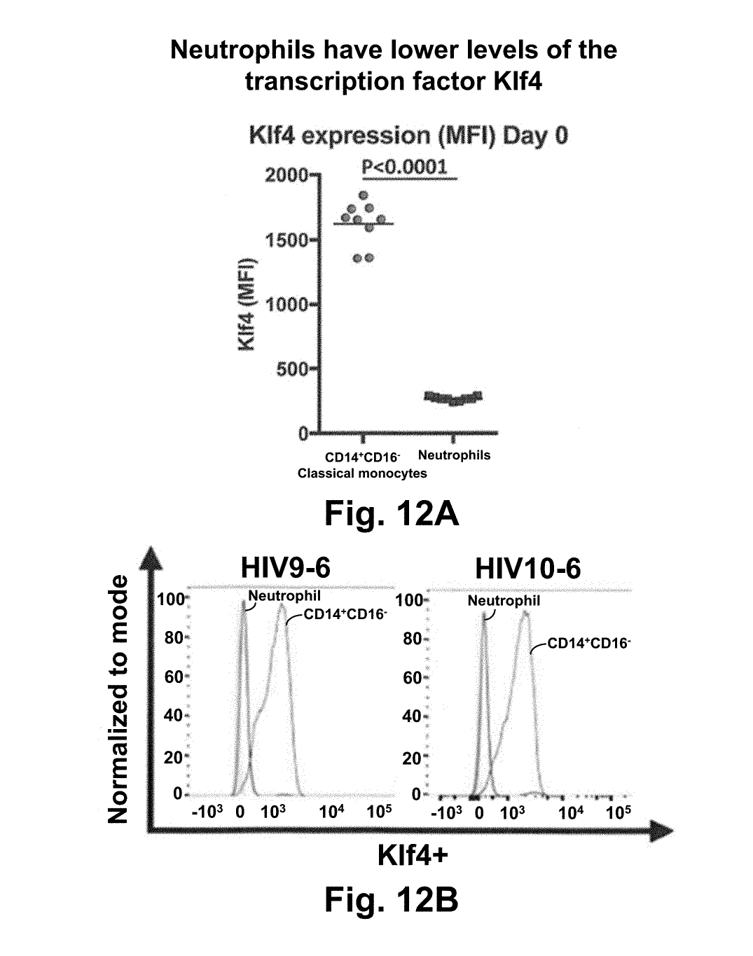

FIGS. 12A and 12B show that Klf4 is more highly expressed in classical monocytes than neutrophils. Loss of Klf4 has been shown to include a M1 phenotype in human macrophages. Neutrophils are very pro-inflammatory, which may be due to Klf4.

SLAMF7 is Upregulated in Response to IFNα in Total PBMCs and Monocytes from HIV+ Individuals.

To examine whether SLAMF7 upregulation may be the result of chronic immune activation, PBMCs are stimulated from HIV+ patients with IFNα, known to be chronically elevated in HIV+ patients. A minor, but significant increase in the percent of SLAMF7 PBMCs is observed following IFNα stimulation as displayed in FIGS. 6E and 6F. To identify if a specific cell type is responsible for this increase in SLAMF7⁺ cells, the SLAMF7^{high} peak present in IFNα samples and not in mock treated is gated on and various cell markers are looked at. Greater than 95% of the cells in this SLAMF7^{high} peak are CD14⁺ monocytes as shown in FIGS. 6G-6I. Looking at just CD14⁺ monocytes, it is found that they show the most robust SLAMF7 response to IFNα as displayed n FIGS. 6J and 6K. Since monocytes play a critical role in type I interferon responses and are implicated in the pathogenesis of a number of secondary HIV-associated pathologies, what role SLAMF7 may be playing in monocytes is assessed.

SLAMF7 Activation on Monocytes of HCs Inhibits IFNα-Mediated CXCL 10 Production.

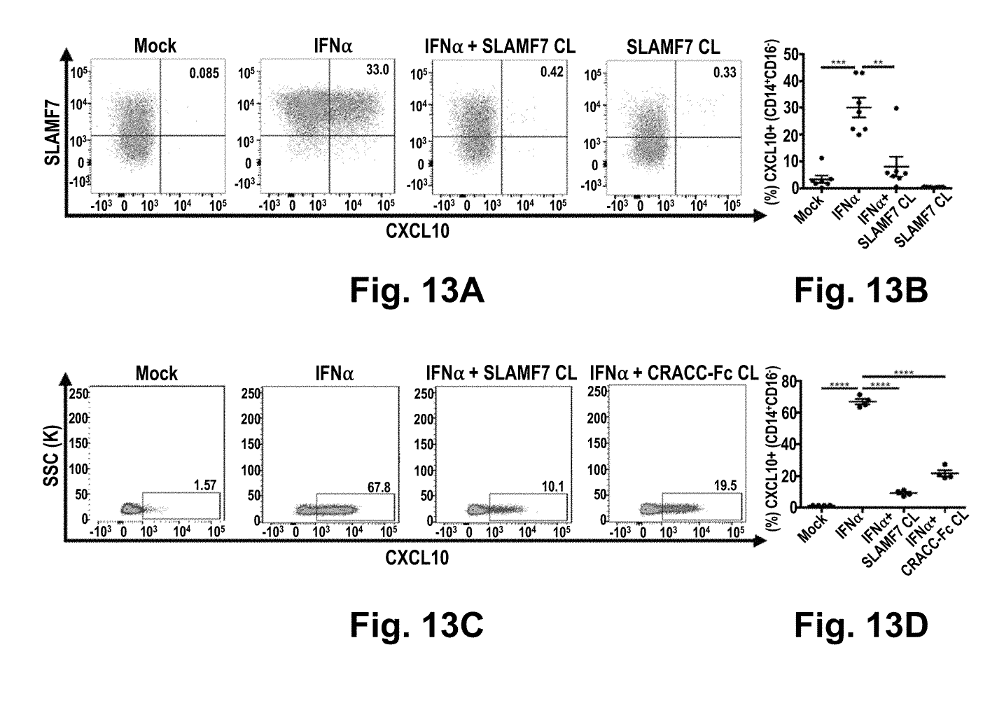

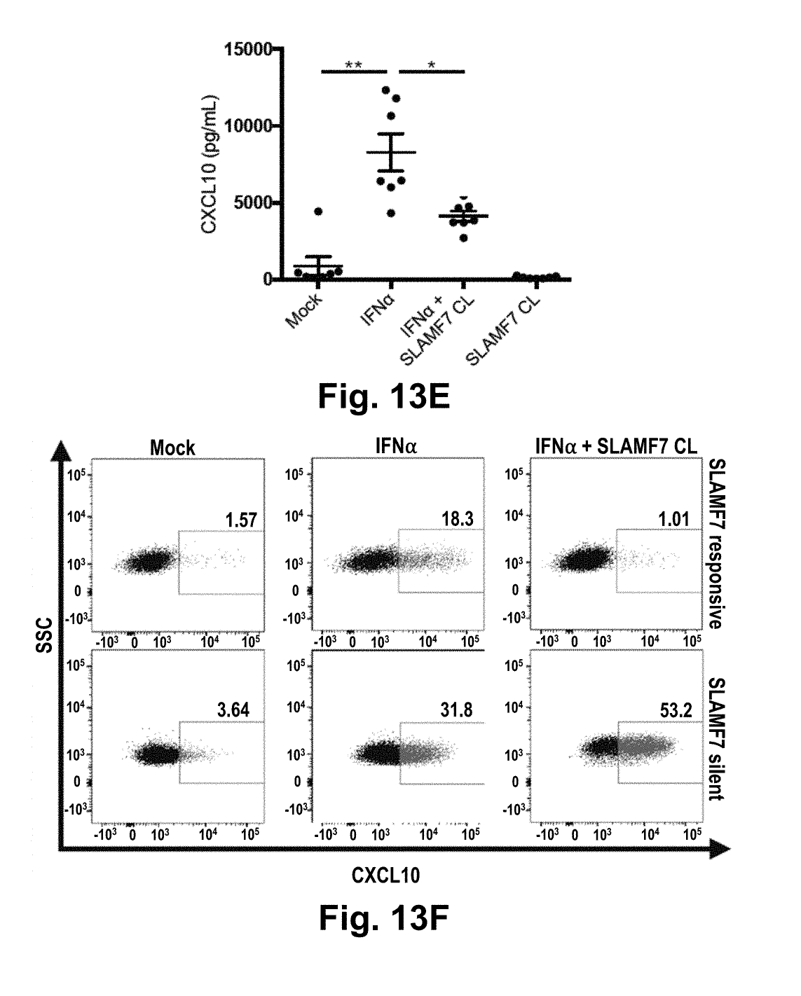

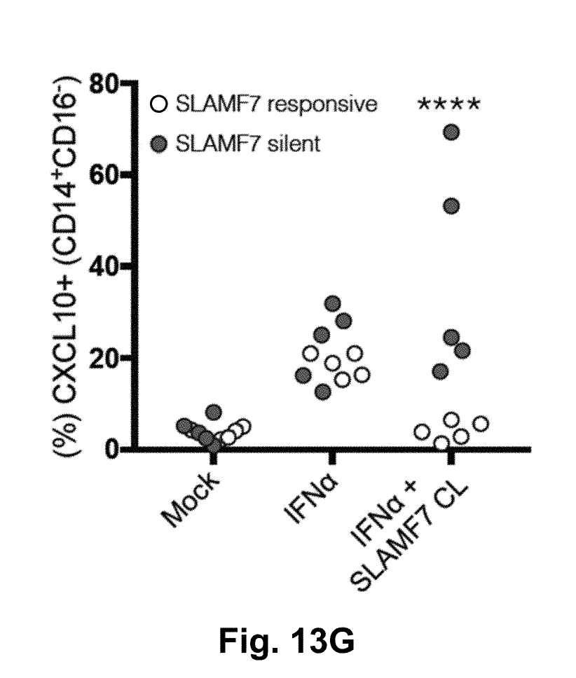

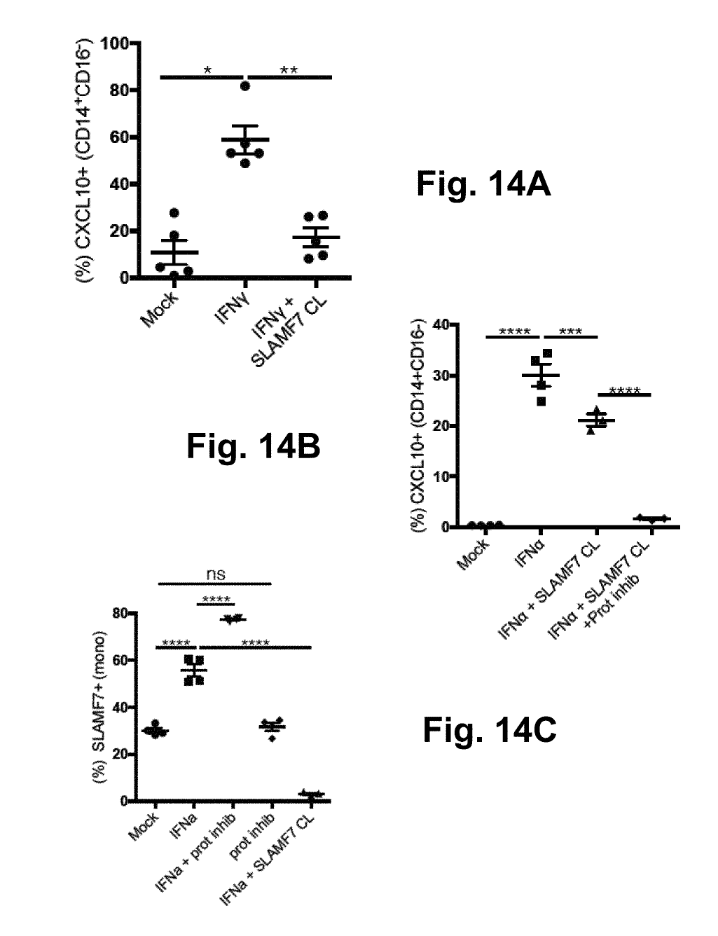

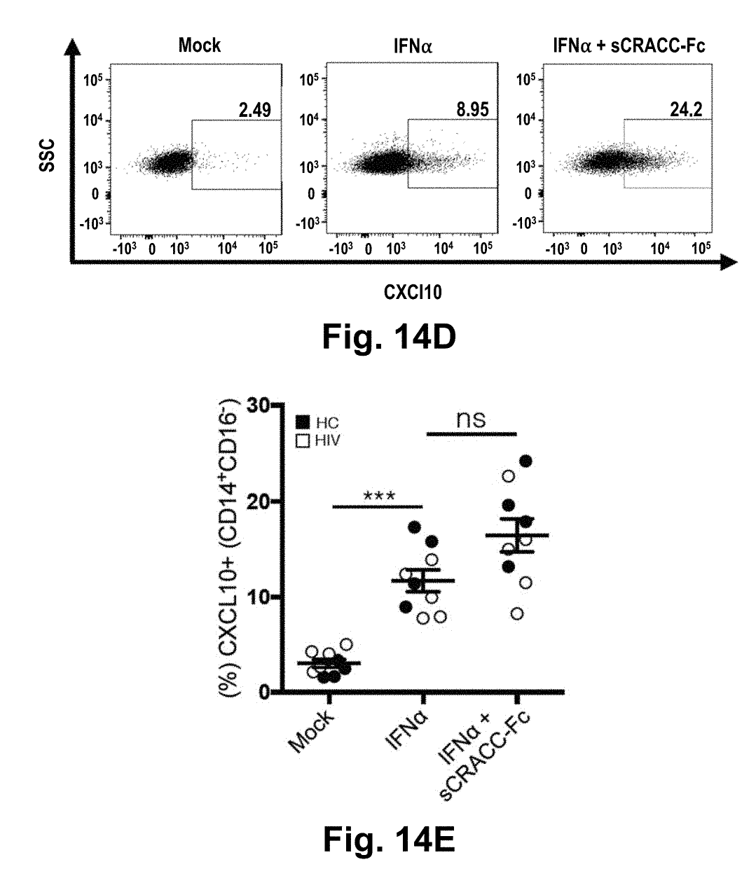

Monocytes are the primary source of CXCL10, especially in response to type I and II interferons. Therefore, the effect of SLAMF7 signaling on IFNα and IFNγ-stimulated PBMCs from HCs is examined. Activation of SLAMF7 during IFNα as shown in FIGS. 13 and 13B and IFNγ-stimulation as shown in FIG. 14A result in robust inhibition of CXCL10 production from monocytes. To confirm this effect is specific to monocytes, primary CD14⁺ monocytes are isolated and the previous experiment is repeated, noting the same effect as shown in FIGS. 13C and 13D. To verify that CXCL10 inhibition is SLAMF7-specific, SLAMF7 is activated via cross-linking with a recombinant version of the SLAMF7 extracellular domain, SLAMF7-Fc. FIGS. 13C and 13D show the final condition. Monocytes are also treated with soluble SLAMF7-Fc in vitro, confirming that binding of cell surface SLAMF7 to an immobilized SLAMF7 extracellular domain is necessary for inhibition as shown in FIGS. 14D and 14E. Furthermore, it is confirmed that activation of SLAMF7 results in reduced secretion of CXCL10 from IFNα-stimulated PBMCs into the culture supernatant via BioPlex assay as shown in FIG. 13E. However, SLAMF7 activation did not robustly reduce supernatant levels of CXCL10 to the degree that is observed with flow cytometry, suggesting the putative involvement of other cell type(s).

A Subset of HIV+ Patients are Non-Responsive to SLAMF7 Inhibitory Signaling.

It is next examined if this effect is conserved in HIV+ individuals. The same experiment performed in FIGS. 13A and 13B is repeated with freshly isolated PBMCs from HIV+ patients. A dichotomous response in HIV+ patients is noted, with some responding the same as HCs (referred to hereafter as “SLAMF7 responsive”) and some showing a failure to inhibit CXCL10, which is hereafter referred to as “SLAMF7 silent” (SF7S) as shown in FIGS. 13F and 13G. As an internal control to verify that these differences are not due to experimental variability, HCs are included alongside HIV+ samples in each experiment and it is noted that in each case, all HCs did respond to SLAMF7 activation. Defining SLAMF7 as an inhibitory receptor in monocytes and identifying a subset of HIV+ patients with a defect in SLAMF7 signaling leads to the examination of if there is any correlation to clinical biomarkers associated with chronic immune activation.

SLAMF7 Response and SF7S Patients have Distinct Peripheral Immune Activation Signatures.

The plasma levels of six proinflammatory cytokines and chemokines implicated in chronic immune activation during HIV infection are evaluated. Hierarchal and k-means clustering is used to identify patients with similar peripheral immune activation profiles. Hierarchal clustering identifies two distinct clusters and FIG. 15A shows the results of k-means clustering. Cluster one is characterized by low levels of all six proinflammatory factors, while Cluster two shows patients with elevated levels of all six proinflammatory factors. A heatmap of plasma cytokines and chemokines shows Cluster one patients exhibiting a similar profile to that of HCs, while Cluster two patients have a markedly different cytokine profile as displayed in FIG. 15B. Comparison of demographics and clinical characteristics show the two clusters to be otherwise well balanced as shown in Table 2 below. Interestingly, it is found that Cluster 2 patients are all SF7S while Cluster 1 patients are predominantly SLAMF7 responsive as shown in FIG. 15C. These results suggest that dysfunction of the SLAMF7 receptor may result in in vivo manifestations.

TABLE 2
Cluster Characteristics

| | Cluster 1 | | Cluster 2 | |
|---|---|---|---|---|
| n | 28 | | 12 | |
| Median age, (IQR) | 51 | (17.25) | 54 | (6.25) |
| Median BMI, (IQR) | 28.5 | (5.78) | 28.75 | (12.9) |
| Race- n, (%) | | | | |
| Caucasian | 17 | (60.7) | 7 | (58.3) |
| African | 8 | (28.6) | 3 | (25) |
| Hispanic | 3 | (10.7) | 1 | (8.3) |
| Unknown | 0 | | 1 | (8.3) |
| Sex- n, (%) | | | | |
| Male | 21 | (75) | 10 | (83.3) |
| Female | 7 | (25) | 2 | (16.7) |
| Median CD4 count (cells/μL), (IQR) | 591.5 | (500) | 688 | (402) |
| Median CD4/CD8 ratio, (IQR) | 0.61 | (0.96) | 0.45 | (0.82) |
| CD4 nadir (cells/μL)- n, (%) | | | | |
| <50 | 7 | (25) | 4 | (33.3) |
| 50-100 | 5 | (17.9) | 0 | |
| 100-200 | 5 | (17.9) | 4 | (33.3) |
| 200-350 | 3 | (10.7) | 1 | (8.3) |
| 350-500 | 1 | (3.6) | 2 | (16.7) |
| >500 | 7 | (25) | 0 | |
| Unknown | 0 | | 1 | (8.3) |
| Clinical phenotype- n, (%) | | | | |
| Concordant | 20 | (71.3) | 8 | (66.7) |
| Discordant | 3 | (10.7) | 3 | (25) |
| Elite Controller | 4 | (14.3) | 0 | |
| LTNP | 1 | (3.6) | 1 | (8.3) |
| Viral load (copies/mL)- n, (%) | | | | |
| ND | 20 | (71.3) | 8 | (66.7) |
| 20-1000 | 5 | (17.9) | 3 | (25) |
| 1000-2500 | 2 | (7.1) | 1 | (8.3) |
| Unknown | 1 | (3.6) | 0 | |
| Median length of injection (years), | 16.5 | (17.25) | 14 | (22.75) |
| (IQR) | | | | |
| Current MJ use- n, (%) | | | | |
| Yes | 9 | (32.1) | 6 | (50) |
| No | 18 | (64.3) | 6 | (50) |

| | | | |
|---|---|---|---|
| Unknown | 1 | (3.6) | 0 |

Monocytes from SF7S and SLAMF7 Responsive Patients do not Differ in Expression Levels of the SLAMF7 Receptor or EAT-2 Adaptor.

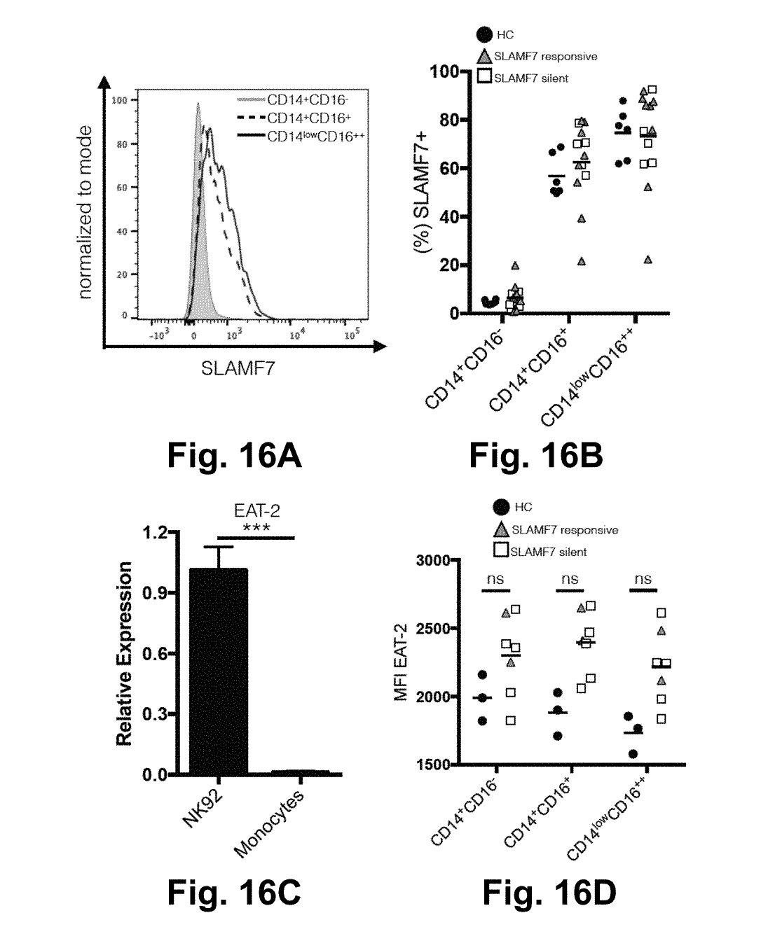

To determine the mechanism behind this defective SLAMF7 function in SF7S patients, levels of SLAMF7 expression across all monocyte subsets in HCs, SF7S, and SLAMF7 responsive patients are assessed. A small percentage of classical monocytes (approximately 5%) express SLAMF7 at baseline, while a significantly higher percentage of both intermediate (approximately 60%) and non-classical monocytes (approximately 70%) express SLAMF7 as shown in FIGS. 16A and 16B. Critically, a difference in SLAMF7 expression between SF7S and SLAMF7 responsive patients is not observed, as shown in FIG. 16B. Since it is well established that the presence or absence of the SLAM family adaptor EAT-2 can dramatically alter SLAMF7 signaling and govern the activating function of SLAMF7, the levels of EAT-2 across all three monocyte subsets in HCs, SF7S, and SLAMF7 responsive individuals are next observed as shown in FIG. 16D. However, it is first examined whether monocytes from HCs express any EAT-2 at all. Comparison of EAT-2 mRNA levels between monocytes and a human NK cell line known to express EAT-2 (NK92), show that monocytes have little to no EAT-2 as shown in FIG. 16C. This result confirms previous findings and partially explains why SLAMF7 acts in a purely inhibitory manner in monocytes. Comparison of EAT-2 at the protein level via flow cytometry showed that all HIV+ individuals have slightly increased EAT-2 expression over HCs as shown in FIG. 16D. However, differences in EAT-2 expression between SF7S and SLAMF7 responsive patients are not observed. Together, these results suggest that the mechanism underlying the loss of SLAMF7 inhibitory activity in monocytes is independent of SLAMF7 and EAT-2 expression levels.

SLAMF7 Signaling in Monocytes Selectively Inhibits IFNα-Mediated Production of Alpha Chemokines Over Other Interferon-Stimulated Genes (ISGs) and Host Restriction Factors.

Next, it is investigated if the robust inhibition of CXCL10 is conserved across other alpha chemokines and ISGs. The inhibition of CXCL10 is conserved at the mRNA level and this effect is consistent across CXCL9, CXCL11, and CXCL12 as shown in FIG. 17A. Surprisingly, a number of other interferon stimulated genes and HIV-associated, interferon-modulated, host restriction factors including: BST2, OAS1, Trex1, and STAT1 show only minor or no inhibition following SLAMF7 activation as shown in FIG. 17B.

SLAMF7-Mediated Inhibition of CXCL10 Production in Monocytes May be Independent of SHP1, SHP2, SHIP1, or CD45.

To better understand the mechanism behind SLAMF7's inhibitory functions in monocytes, a number of inhibitory phosphatases previously shown to interact with SLAMF7 and mediate its inhibitory functions are evaluated. In an effort to continue using primary human monocytes, a number of small molecule inhibitors targeting SHP1, SHP2, and SHIP1 are tested. Pre-treatment with either a small molecule inhibitor of SHP1/2 (NSC87877) as shown in FIGS. 17A and 17C or SHIP1 (3AC) as shown in FIG. 17C does not result in recovery of CXCL10 expression in monocytes stimulated with IFNα and SLAMF7 cross-linking. It is hypothesized that SLAMF7's inhibitory function is mediated by CD45, as CD45 has been previously implicated in SLAMF7's ability to propagate inhibitory signals in NK and multiple myeloma cells. Pre-treatment with a small molecule inhibitor specific for CD45 (CAS 345630-40-2) in the presence of IFNα and SLAMF7 cross-linking also fails to rescue CXCL10 production as shown in FIG. 17D. Finally, SLAMF7 might prevent CXCL10 production by a mechanism that involves CXCL10 proteasomal degradation. Addition of the proteasome inhibitor Bortezomib does not result in recovery of CXCL10 expression in SLAMF7-stimulated cells, and actually further decreases CXCL10 production as shown in FIG. 14B. Interestingly, it is noted that Bortezomib acts synergistically with IFNα to upregulate SLAMF7 expression on monocytes as shown in FIG. 14C.

SLAMF7 Inhibits Monocyte Infection with HIV-1 In Vitro.

HIV+ patients are known to have increased levels of circulating inflammatory monocytes (CD14⁺CD16⁺ and CD14^{low}CD16⁺⁺), which promote peripheral immune activation, can become infected with HIV virus, and induce HIV-associated neurocognitive disorder (HAND). Therefore, it is next looked at if SLAMF7 activation can affect the ability of monocytes to transition into pro-inflammatory subtypes (CD16⁺), as well as monocyte susceptibility to HIV virus infection. Monocyte infection with HIV occurs via CCR5, which is known to be upregulated following IFNα stimulation. It is discovered that activation of SLAMF7 on monocytes from HIV+ individuals can down-regulate CCR5 both in the presence and absence of IFNα as shown in FIGS. 18A and 18B. Further supporting SLAMF7's utility as a means to prevent monocyte infection, activation of SLAMF7 in the presence of IFNα significantly upregulates the CCL3L1 chemokine, a chemokine that binds to CCR5 and directly prevents HIV infection of monocytes, as shown in FIG. 18C. Based on these findings, an in vitro infection of isolated primary human monocytes from 2 HCs is performed to determine if SLAMF7 activation could prevent HIV-1 infection. Infection of monocytes with HIV-1-_{Ba-L}-GFP in the presence of SLAMF7 cross-linking results in dramatically reduced percentages of HIV-1 infected cells as shown FIG. 18D. To determine if SLAMF7 signaling affects CD16 expression on monocytes, a IFNα-driven monocyte maturation assay is utilized as previously described. A significant down-regulation of CD16 on monocytes with SLAMF7 activation is observed both in the presence and absence of IFNα stimulation as shown in FIG. 18E, suggesting that SLAMF7 signaling can prevent induction of inflammatory monocyte subsets.

Neutrophils Constitutively Express CXCL10 and do not Express SLAMF7.

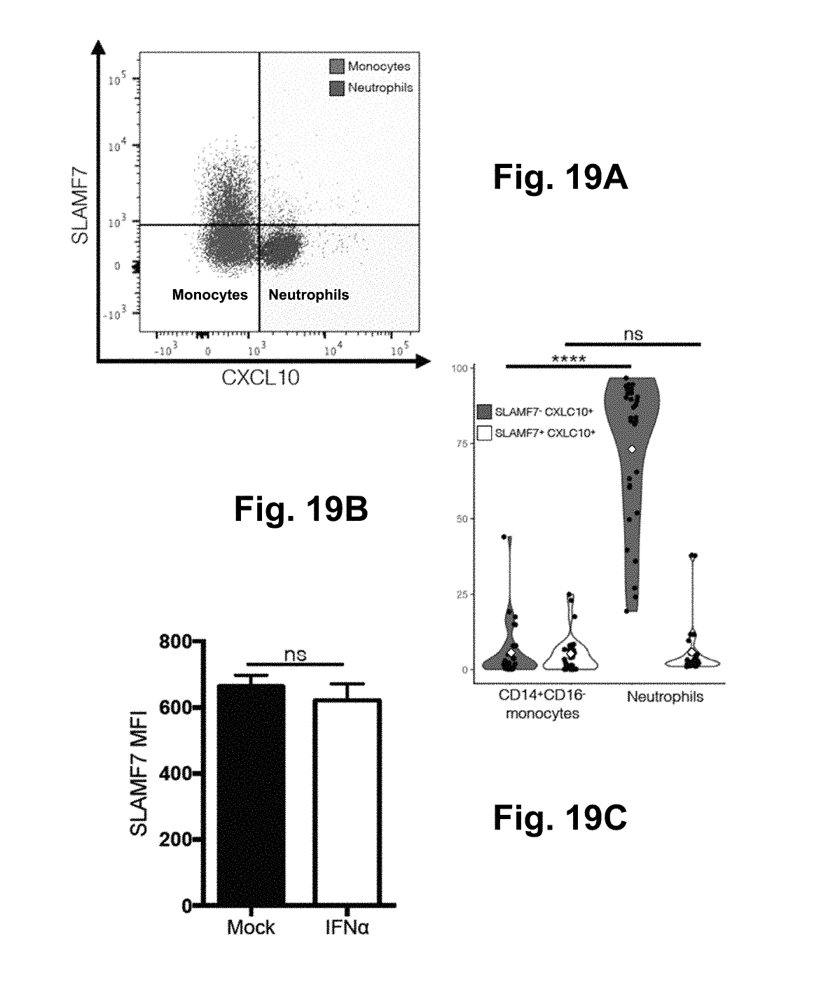

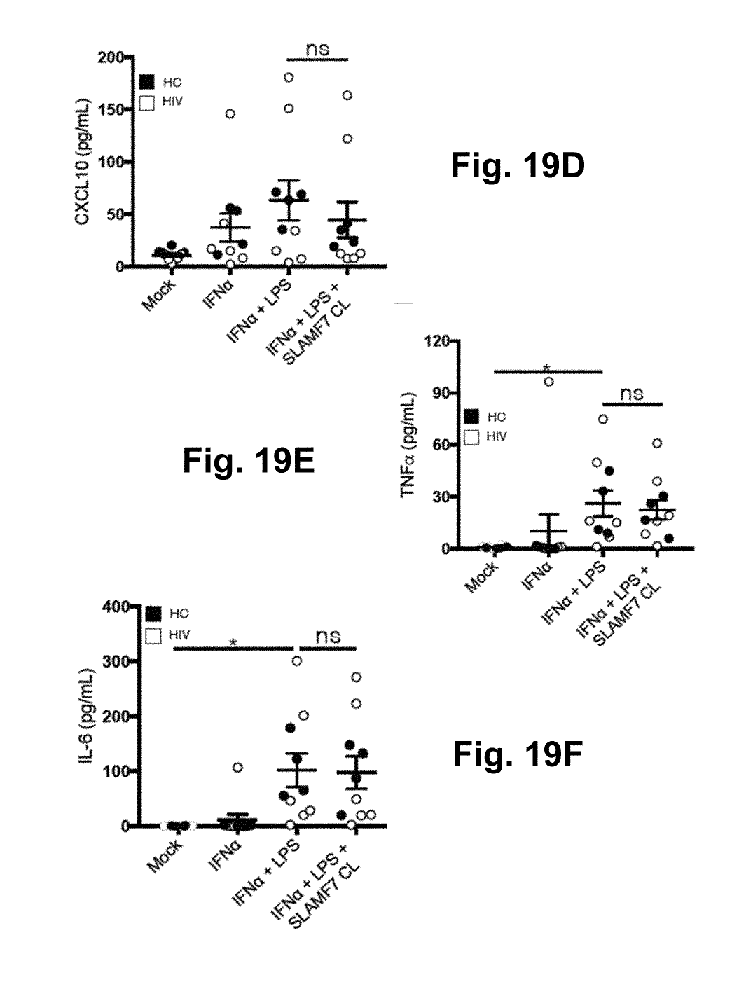

Upon further review of the in vitro data, a peculiar discrepancy between the flow cytometry and BioPlex results is observed. Comparing FIGS. 13B and 13E, it is noted that while near complete inhibition of CXCL10 from IFNα-stimulated monocytes is observed following SLAMF7 activation on FACS analysis, just an approximately 50% reduction in CXCL10 concentration in the culture supernatant is shown. While this could have been from residual buildup of CXCL10 in the supernatant that occurs before SLAMF7 inhibition takes effect, it has been determined that SLAMF7 begins to exert its inhibitory effects within a very short time frame (less than 4 h), based on isolated monocyte experiments as shown in FIG. 13D. This suggests that another cell type may also be responsible for CXCL10 production and may not be responding to SLAMF7 activation. Neutrophils (CD66b⁺ CD3⁻ CD19⁻ CD57⁻ CD16⁺⁺ CD14^{low/-}) are identified as being SLAMF7⁻ and CXCL10⁺ at baseline in cART treated HIV individuals as shown FIGS. 19A and 19B.

Neutrophils are Unable to Upregulate SLAMF7 in Response to IFNα and SLAMF7 Activation does not Inhibit Proinflammatory Cytokine and Chemokine Release from Neutrophils.

SLAMF7 expression on neutrophils in the presence and absence of IFNα stimulation is assessed by flow cytometry. It is observed that neutrophils are unable to upregulate SLAMF7 in response to IFNα as shown in FIG. 19C. This, combined with the knowledge that they are CXCL10⁺ at baseline, suggests that following appropriate stimulation, they should constitutively release CXCL10 regardless of the presence of SLAMF7 activating mAbs. This is tested via BioPlex analysis, utilizing freshly isolated neutrophils from both HCs and HIV+ patients. It is discovered that IFNα by itself can induce CXCL10 release from neutrophils, that this effect is enhanced by addition of LPS, and that the addition of SLAMF7 cross-linking mAbs very minimally decreases CXCL10 release as shown in FIG. 19D. Consistent with this result, a similar pattern is noted in regards to TNFα and IL-6 release from IFNα and LPS co-stimulated neutrophils as shown in FIGS. 19E and 19F, respectively.

Discussion

The discovery that global levels of SLAMF7⁺ PBMCs are increased in HIV infected patients in a clinical phenotype-dependent manner suggests that SLAMF7 receptor functions play a role in modulating peripheral immune activation. Supporting the idea that SLAMF7 is a marker of elevated type I interferons are reports showing SLAMF7 to be upregulated in systemic lupus erythematosus, rheumatoid arthritis, and multiple sclerosis. While the effects of IFNα on SLAMF7 expression are focused on here, it is well known that LPS can also up-regulate SLAMF7 through a NF-κB-dependent mechanism. HIV+ patients are known to have elevated levels of LPS in their blood as a result of a “leaky gut”; this cannot be excluded as an additional possible reason for elevated SLAMF7 levels. The findings that discordant patients have extremely elevated SLAMF7 levels and that elite controllers have levels of SLAMF7 comparable to healthy controls supports the idea that assessing SLAMF7 expression levels on peripheral immune cells could be an effective gauge of a patients' immune-activation status.

The finding that SLAMF7 activation can specifically inhibit CXCL10 and other alpha chemokines in monocytes is consistent with previous studies showing SLAMF7 has inhibitory effects in LPS-stimulated monocytes. Interestingly, SLAMF7 activation minimally reduces HIV restriction factors; thus, pharmacological modulation of SLAMF7 in the context of HIV infection could be beneficial in reducing peripheral immune activation and preventing monocyte infection, without affecting important viral restriction factors. However, this approach may only be effective in some HIV patients, since a subset of HIV+ individuals are identified, SF7S, who show a lack of response (or inverse response) to SLAMF7 activation. It is possible that the underlying mechanism to this paradoxical lack of SLAMF7 response is due to either genetic differences between patients, differential alterations in circulating cytokines or chemokines, or from specific interactions with HIV viral proteins.

The small molecule studies suggest that there are yet other, unidentified inhibitory factor(s) which can interact with SLAMF7, or SLAMF7 may propagate its signals through ITSM-independent mechanisms. Supporting this is the fact that most of the studies regarding the interaction of SLAMF7 with inhibitory phosphatases are performed in mice or human cell lines; thus, it is possible that some of these findings are not entirely translatable to primary human cells. Regardless, SLAMF7 activation in monocytes may prove to be a useful method of preventing pathological activation of the CXCR3 receptor since it inhibits all CXCR3 ligands.

Both clinical and pre-clinical attempts at CXCL10 neutralization and/or CXCR3 receptor blockade have largely failed thus far. Reasons for this include: failure of CXCL10− specific mAbs to inhibit all CXCR3 ligands, inability of anti-CXCL10 mAbs to compete with the high synthesis and turnover rate of CXCL10, and inability of some anti-CXCL10 mAbs to bind the glycosaminoglycan (GAG) bound form of CXCL10 (the active form of CXCL10). Importantly, SLAMF7-mediated inhibition of alpha chemokines has the ability to overcome all of these limitations and may find itself to be a useful therapeutic modality in diseases where over-expression of CXCL10 has been linked to pathogenesis including: rheumatoid arthritis, type I diabetes mellitus, systemic lupus erythematous, multiple sclerosis, ulcerative colitis, and primary biliary cirrhosis.

It is well established that patients with HIV or a number of other diseases have elevated levels of CD16⁺ monocytes and that these pro-inflammatory cells have been implicated in the pathogenesis of these conditions. There are currently no methods to reduce the number of circulating CD16⁺ monocytes in these individuals, except for a single in vitro report studying one of the components of cannabis, THC. It would be interesting to see if SLAMF7-mediated down-regulation of CD16 is consistent in vivo and, if so, if there is any resultant effect on pathology in pre-clinical animal models.

Similar to its effect on CD16, the down-regulation of CCR5 on monocytes by SLAMF7 signaling could have potential benefits in the setting of HIV infection. While there have been numerous attempts at either blocking/down-regulating CCR5 or increasing the levels of chemokines, such as CCL3L1, (or engineered chemokines) specific for CCR5 for the treatment of HIV infection, there have not been any attempts to simultaneously apply both approaches. Activation of SLAMF7 on monocytes is unique in that it can accomplish this and potentially inhibit HIV-1 infection of monocytes and other CCR5-expressing immune cells.

The results show that neutrophils are SLAMF7⁻ and CXCL10⁺ at baseline and are able to respond to stimulation by type I interferons, but not to SLAMF7 activation. The role of neutrophils in the context of HIV infection is complex and understudied. Specifically, whether or not neutrophils contribute to peripheral immune activation in cART-treated individuals is unclear. The results suggest that neutrophils likely do contribute to peripheral immune activation. Additionally, neutrophils are also known to play an important role in neuroinflammation and have recently been discovered to be present in high levels in the brain at steady state. While the role of neutrophils in the CNS of HIV+ individuals and in HAND is unknown, the results suggest that if they do play a role, it is likely one that cannot be modulated through the SLAMF7 receptor. The discovery that neutrophils are able to secrete CXCL10 following IFNα and LPS stimulation highlights the need to consider the effects neutrophils play in diseases characterized by chronic type I interferon activation.

In summary, SLAMF7 is up-regulated globally in HIV patients who have high levels of peripheral immune activation and SLAMF7 functions to both prevent HIV viral infection of monocytes and inhibit CXCL10 from monocytes stimulated with type I and II interferons, except in a subset of HIV patients. It is also discovered that neutrophils fail to express SLAMF7, constitutively express CXCL10, and are non-responsive to SLAMF7 activation, implicating them as potential propagators of chronic, peripheral immune activation in type I interferon-mediated diseases.

### Example 2

SLAMF7 Acts as a Critical Negative Regular of Alpha Chemokines in Monocytes and Neutrophils from HIV+ Individuals Via a YY1-Dependent Mechanism.

Chronic immune activation, characterized by sustained levels of IFNα, LPS and other immune activators, is a hallmark of HIV infection. While current cARTs excel in controlling viremia, they fail to fully control excessive immune activation in patients and as a result a number of secondary diseases arise in patients on long term cART. One of these diseases, HIV associated neurocognitive disorder (HAND), results in dementia-like symptoms in up to 50% of patients. HAND is thought to occur through a complex cascade of events that results in increased levels of pro-inflammatory cytokines and chemokines in the CNS, mediated by monocytes, and eventually resulting in neuronal cell death. Of these pro-inflammatory cytokines and chemokines, CXCL10 (IP-10) has been identified as one of the primary drivers of both neuronal cell death and recruitment of immune cells to the brain in HAND and other neurological disorders. Efforts to prevent the negative effects of CXCL10 have thus far failed in both pre-clinical and clinical settings. The ability to robustly inhibit the production of CXCL10, and related chemokines, from monocytes by activating the SLAMF7 (CRACC) receptor is reported. Interestingly, neutrophils are SLAMF7 negative and are a source of CXCL10. Further shown is that this down-regulation of SLAMF7 is through a mechanism involving YY1 acetylation.

Introduction.

SLAMF7 is a member of the SLAM family of receptors and is present on most immune cells. The effects of SLAMF7 have been best characterized in NK cells and can be summed up as being activating in cells containing the SLAM-family adaptor EAT-2 and inhibitory in cells lacking EAT-2. Regulation of SLAMF7 expression has previously been linked to NF-κB, which is confirmed here by showing that interferon-a is able to induce SLAMF7 expression on monocytes (FIG. 6K). Assessing the overall levels of SLAMF7 on total PBMCs from HIV+ individuals, it was found that SLAMF7 levels correlated with HIV-1 clinical phenotypes and by extension, their levels of immune activation (FIG. 6A). This leads to further investigation into what role SLAMF7 may be playing in HIV.

Materials and Methods.

PBMCs are isolated using Ficoll-Plaque PLUS, stained or stimulated in 96-well plates for 12 hrs before the addition of GolgiPlug for 5 hrs. Cultured cells are surface stained and intracellular stained using BD Fixation and Intracellular Staining Kit per manufacturer instructions. Cells are analyzed on either a BD FACS Canto or BD LSR II. Supernatant levels of CXCL10 are determined by running a 27-plex BioPlex assay on the supernatant from in vitro cell culture experiments. For qRT-PCR experiments: CD14+ cells are isolated from fresh human PBMCs using the MACS human CD14 positive selection kit per manufacturer instructions, plated in a 96 well plate, stimulated with 100 IU/mL IFNα and picked up with Trizol. RNA is reverse transcribed with SuperStrand First Strand Synthesis Kit III (Invitrogen) and qRT-PCR reactions are analyzed on a QuantStudio7 system. Intranuclear staining for YY1 is performed using BD Transcription Factor Staining Buffer Set per manufacturer instructions.

Results and Discussion.

In vitro stimulation of primary human PBMCs with 100 IU/mL IFNα (Peprotech) is performed with or without SLAMF7 cross-linking. Both a commercially available monoclonal antibody against SLAMF7 and a proprietary CRACC-IgG4 fusion protein are used to confirm that CXCL10 inhibition is SLAMF7 specific. Additionally, in vitro stimulations are carried out with isolated monocytes, where the same effect is noted (data not shown). Notably, CXCL10 levels in the supernatant of cultured cells did not reach the same level of inhibition seen in intracellular staining (compare FIGS. 13B and 13E), suggesting a second cell type may be responsible for secretion of CXCL10.

It is known that in NK cells lacking EAT-2, SLAMF7 plays an inhibitory role; therefore, the expression of EAT-2 in classical monocytes is observed. Comparison to a known EAT-2 expressing NK cell line confirms lack of EAT-2 expression in monocytes (FIG. 16C). This suggests that SLAMF7 is inhibiting alpha chemokines through recruitment of one of a number of inhibitory phosphatases including: SHP1, SHP2, SHIP1 or csk. In vitro experiments utilizing a small molecule inhibitor of SHP1/2 suggest that these two phosphatases are not involved in alpha chemokine inhibition (FIGS. 17A-17B).

Conclusion.

Within the past few years it has become increasingly clear that chemokines play a critical role in immune regulation and neuroinflammation, yet methods to control their production have largely failed. One proposed reason for the failure of methods to inhibit CXCL10 is the fact that CXCL9 and CXCL11 are also able to bind to the CXCL10− receptor (CXCR3), therefore, a pan-blockade of all three chemokines is needed. It is shown that simply activating the SLAMF7 receptor on monocytes results in a robust pan-inhibition of IFNα-induced alpha chemokines. Pharmacological modulation of this pathway could be of potential benefit in the treatment of a number of neuroinflammatory disorders, including HAND.

The data also suggests that neutrophils may be a significant source of CXCL10 in HIV patients. Previous reports have implicated monocytes as the primary culprit responsible for elevated levels of CXCL10 in the serum and CSF of HIV patients with HAND. Further studies will be required to better elucidate the role of neutrophils in HAND and to determine if re-expression of SLAMF7 on neutrophils is possible and of any benefit.

### Example 3

Modulation of SLAMF7 in Microglia.

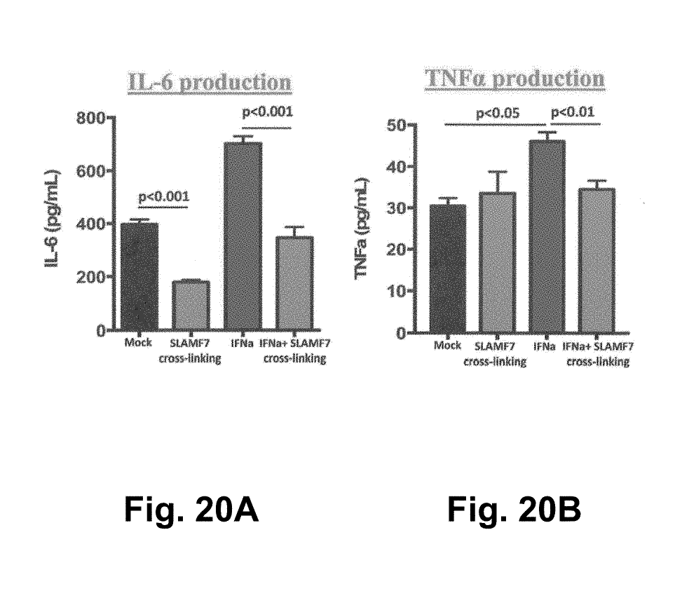

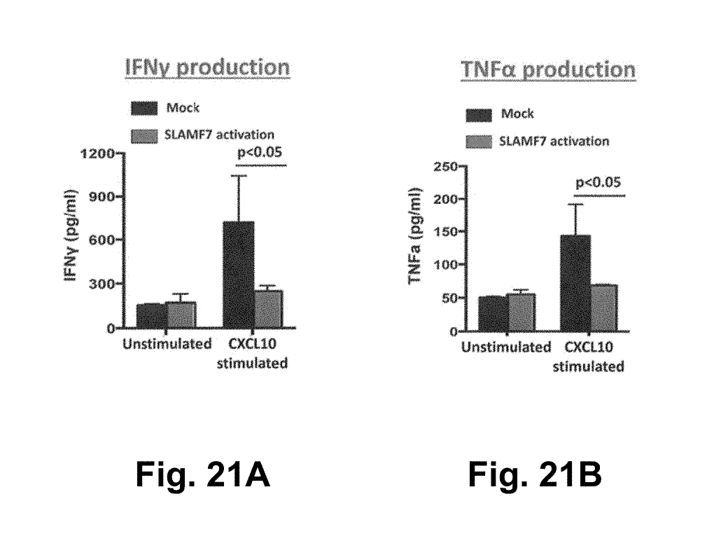

As shown in FIGS. 20A and 20B, investigating the role of SLAMF7 in a human microglia cell line (HMC3) reveals that SLAMF7 activation completely inhibits the production of the proinflammatory cytokines IL-6 and TNFa from these cells, both at the baseline and following type I interferon, LPS, poly(I:C), and recombinant CXCL-10 protein stimulation. For example, human microglia cells are either unstimulated or stimulated with CXCL-10 (30 ng/ml) in the presence or absence of SLAMF7-specific crosslinking protein. After 24 hours, cell supernatants are collected and IFNγ and TNFα production is evaluated by multiplex analysis. Cells are plated in quadruplicate. The data is shown in FIGS. 21A and 21B, where bars represent the SEM. Statistical analysis is completed by using the GraphPad prism 7 software.

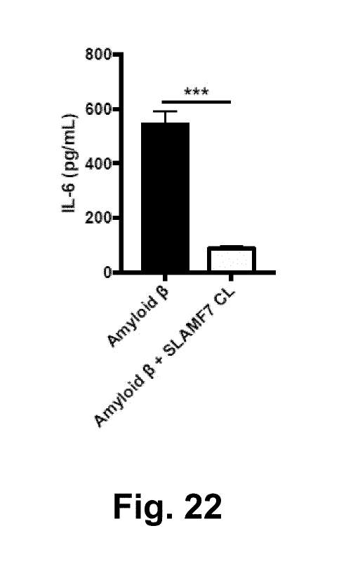

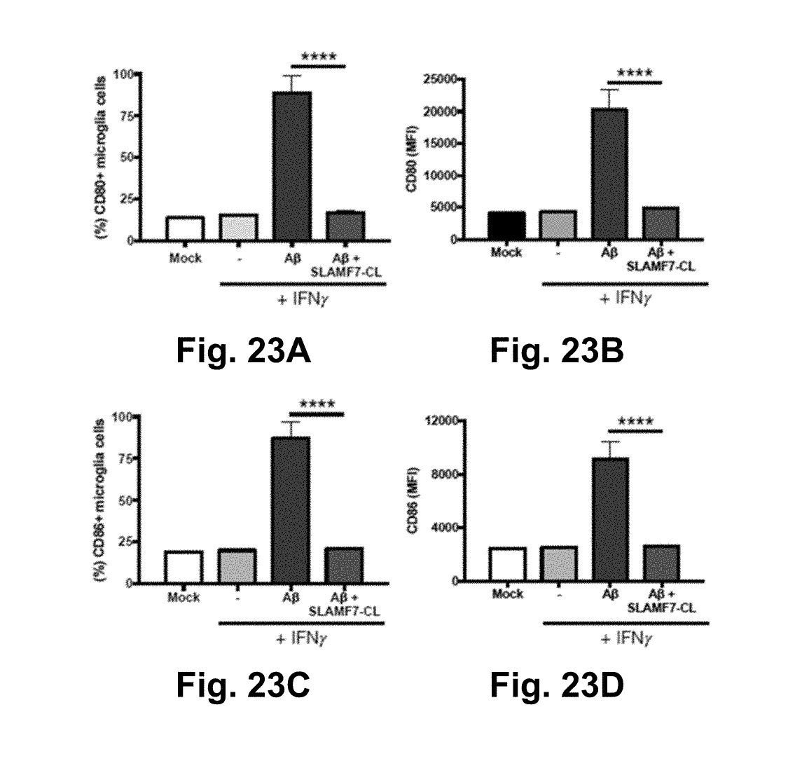

As shown in FIG. 22, SLAMF7 activation inhibits IL-6 production from amyloid β (Aβ)-stimulated human microglia cell line (HMC3) cells. FIGS. 23A-23D show that SLAMF7 activation inhibits CD80 and CD86 co-stimulatory receptor expression from Aβ-stimulated HMC3 cells.

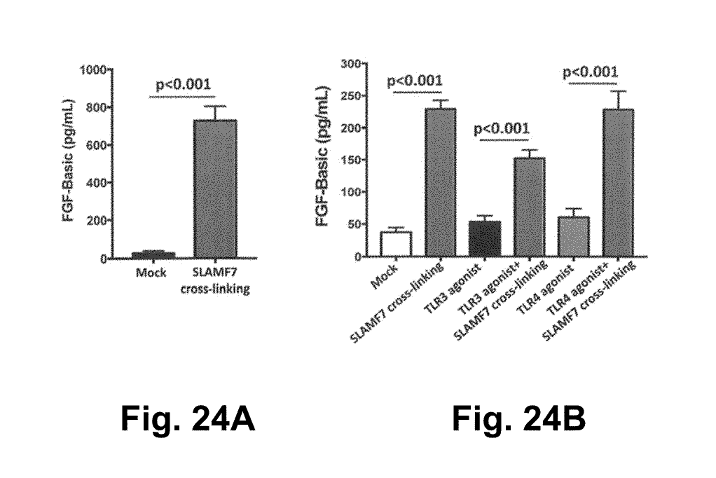

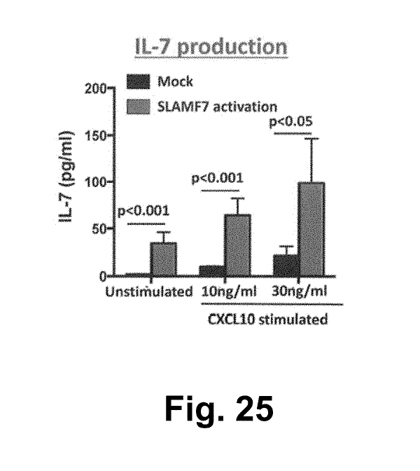

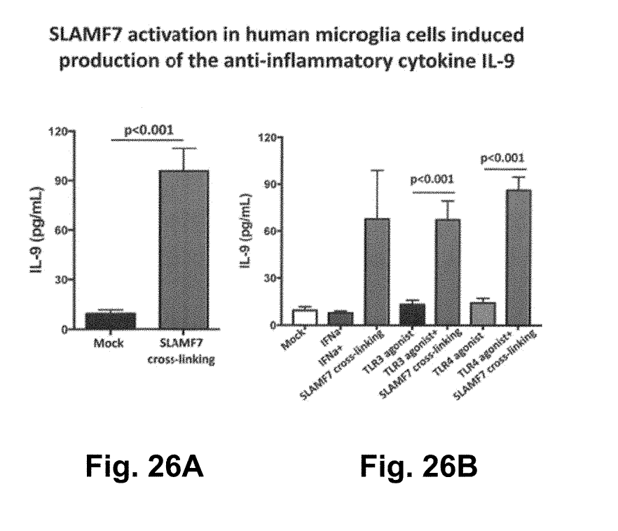

Surprisingly, it is also found that SLAMF7 activation in microglia cells induces a significant amount of the neuroprotective growth factors FGF-basic (see FIGS. 24A and 24B), the Glial cell-derived neurotropic factor (GDNF), and the neuronal survival cytokine, IL-7 (see FIG. 25). As shown in FIGS. 26A and 26B, these responses are also associated with increased levels of the anti-inflammatory cytokine IL-9, a cytokine that is known to promote neuroprotective responses and neuronal survival.

### Example 4

Modulation of SLAMF7 Receptor Signaling in Immune Cells as a Novel Immune Therapeutic Strategy to Treat Autoimmune Diabetes.

Type 1 diabetes (T1D) is an autoimmune disease characterized by progressive immune cell-mediated destruction of insulin-producing β cells. Progress has been made on identifying the role of innate and adaptive immune cells in β cell autoimmunity, but there are no clinical therapies available to prevent T1D in humans. Chemokine-specific monotherapies have been shown to partially prevent the onset of T1D, suggesting that novel therapies targeted to modulate multiple chemokine pathways may be more clinically useful at preventing T1D. Activation of SLAMF7 (a.k.a. CRACC) signaling in human monocytes prevents interferon-stimulated production of several chemokines (CXCL9-12), which are key steps in recruiting T cells to sites of inflammation that also occurs during β autoimmunity. This leads to the hypothesis that immune modulation of SLAMF7 signaling using agonistic antibodies or activating small molecules/peptides during early steps of β cell autoimmunity will prevent interferon-mediated activation and recruitment of T cells thereby preventing T1D.

Provided is a novel immunotherapy strategy needed to modulate both the innate and T cell immune responses during early stages of autoimmunity. This approach prevents T1D in both inducible and spontaneous rat models of T1D. Modulation of innate and T cell immune responses during autoimmunity is achieved by targeting a novel immune receptor, the signaling lymphocytic activation molecule family member 7 (SLAMF7). This therapeutic approach is tested in the novel LEW.1WR1 rat in which β cell autoimmunity can temporally be induced. Sequential productions of various innate immune mediators as well as activation and recruitment of innate and adaptive immune cells followed by β cells destruction and T1D development are monitored. The therapeutic approach is tested in the BB rat to show that the approach prevents T1D in a rodent model with high risk of spontaneous T1D.

According to the Juvenile Diabetes Research Foundation (JDRF), there are 1.25M Americans with T1D and 40,000 people are diagnosed annually in the US. Diabetes care related expenses reached $245 billion in 2012 in the US, and is expected to increase to more than $622 billion in 2030. Although there have been significant advances on understanding the etiology of the disease, there are no therapies available to prevent the disease. Thus, there is an urgent need for a novel strategy that prevents T1D, such as concomitant targeting of multiple chemokine pathways that contribute to T1D initiation. Activating SLAMF7 signaling pathways prevent the production of CXCL9, CXCL10/IP-10, CXCL11, and CXCL12 chemokines from interferon alpha (IFNα)-stimulated human monocytes. Additionally, SLAMF7 activation in human CD8+ T cells prevents their activation and proliferation. In an innovative approach, an immune modulation strategy is developed that targets the SLAMF7 signaling in monocytes and T cells of T1D patients. Critically, an altered expression of human chemokines has been associated with or implicated in T cell-mediated destruction of insulin-producing pancreatic β-cells, and eventually the development of T1D. In particular, increased expression levels of the alpha chemokines CXCL9, CXCL10, and CXCL11, and signaling by their corresponding receptor (CXCR3), are detected in the pancreatic lesions of both recently diagnosed and long-standing subjects with type 1 diabetes. Therapeutic strategies that only target a single chemokine/chemokine receptor pathway show partial prevention of T1D in multiple experimental systems, implicating a potential utility of targeting the chemokine/chemokine receptor system. Lack of complete prevention of T1D in chemokine-specific monotherapies indicates the need to concomitantly target multiple relevant chemokines, a strategy that is supported by a recent finding that showed complete prevention of spontaneous T1D in NOD mice as a result of chemokines neutralization by the β-cell-derived decoy receptor. In addition to chemokine targeting, it has been shown recently that type I IFN receptor (IFNAR1) deficiency protected LEW.1WR1 rats from viral and IFNα-induced T1D. This indicates that alteration of interferon-mediated innate immune responses might influence the onset of T1D, an alteration that is observed following SLAMF7 activation during IFNα stimulation of human monocytes and T cells. Here, an immune modulation strategy is developed that prevents chemokine production and T cell recruitment to islet cells, thus preventing the development of T1D. Activating the SLAMF7 signaling pathways avoids the production of multiple chemokines and inhibits the development of autoreactive T cells. The successful development of SLAMF7-targeting immunotherapeutic strategy is a novel T1 D-targeting immunotherapy platform.

SLAMF7 activation prevents type I interferon-dependent chemokine production from monocytes, a critical step in recruiting T cells to sites of inflammation. Additionally, SLAMF7 has a novel role in T cell regulation. It is now shown that activation of SLAMF7 prevents monocyte-mediated recruitment of T cells to pancreatic islets and ultimately prevents β cell autoimmunity. A T1D rat model has been developed to test this hypothesis. In this model, T1D is induced in a unique type I interferon-dependent manner and sequential invasion of pancreatic islets with monocytes followed by T cells is monitored, which ultimately leads to β cell death and T1D. Here, the impact of SLAMF7 activation in diminishing T1D development in this T1D rat model along with a spontaneous T1D rat model is described. Activation of SLAMF7 is achieved by manufacturing and testing multiple SLAMF7 agonistic antibodies. The most efficacious SLAMF7 agonistic antibody is not only a unique platform for T1D treatment but is useful for treating other autoimmune diseases that are characterized by alteration of the innate (chemokines) and adaptive (T cell) immune responses.

Increased production of proinflammatory chemokines has been associated with T1D pathogenesis. Activating SLAMF7 pathway inhibits proinflammatory chemokine production from human monocytes. SLAMF7 activation prevents the production of CXCL9, CXCL10/IP-10, CXCL11, and CXCL12 chemokines from IFNα-stimulated human monocytes. Additionally, SLAMF7 engagement on activated CD8+ T-cells completely inhibits CD107a expression versus control, suggesting that signaling downstream of the SLAMF7 receptor inhibits CD8+ T cytotoxicity. These findings are confirmed in vivo in mice using a murine SLAMF7 blocking Fc fusion protein. As evidence that modulating SLAMF7 impacts β cell autoimmunity and T1D risk, it has been recently shown that blockade of the PD1 receptor in human T cells developed a T1D phenotype in some cancer patients. A similar phenotype was also noted after the use of CTLA-4 blocking antibody. Therefore, activating PD-1 and CTLA-4 pathways is a promising immunotherapeutic strategy for treating autoimmune diseases. Here, it is shown that activating the SLAMF7 signaling pathway in T1D patients' monocytes suppresses proinflammatory chemokines production and minimizes the activation and recruitment of T cells to pancreatic islets. In addition, SLAMF7 activation in T1D patients' T cells inhibits the development of islet antigen-specific T-cell immune responses, thereby inhibiting their destruction and the development of T1D.

SLAMF7-activating antibodies are designed and manufactured (sub-contracted with an antibody-producing vendor, such as Aragen Biosciences and GenScript). The potency of the SLAMF7 agonistic antibodies are evaluated by measuring their ability to block CXCL chemokines production from IFNα-stimulated monocytes. The most potent SLAMF7 antibodies are then be tested in vivo in both inducible and spontaneously developing T1D rat models. To induce T1D phenotype, LEW.1WR1 rats are intraperitoneally (IP) injected every other day with the type I interferon-inducing TLR3 agonist, poly(I:C) (1 μg per g body weight). This induces a sequential invasion of pancreatic islets with CD68+ monocytes followed by CD3+ T cells (insulitis) by 10 days, after which β cell death and T1D occurs within 15 days of treatment. Dose escalation studies are initially performed to identify the optimal dose of the SLAMF7 agonistic antibody that can be utilized. The SLAMF7 antibody, or the isotype control, is concomitantly coadministered (IP) with poly(I:C), and the development of insulitis and T1D is monitored at various time points (day 3, 6, 8, 10, and 15). Early time points are examined in case modulation of SLAMF7 inadvertently promotes accelerated autoimmunity. Innate immune responses (production of Th-1- and Th-2-skewing proinflammatory cytokines and chemokines and the expression of Th-1- and Th-2-inducing innate immune genes) are evaluated by quantitative RT-PCR and multiplex beads assay. Innate immune cells activation (e.g. expression of CD69, CD107a on NK cells) and maturation (expression of CD80, CD86, CCR7, CD40, MHC-II on DCs and macrophages) and recruitment of these cells to the islets are assessed by flow cytometry and immunohistochemistry analysis. Infiltration of monocytes (day 1-6), CD38+ (day 6) and CD3+ T cells (day 6) into the islet parenchyma, the development of insulitis (day 10), and T1D (day 15) are monitored in SLAMF7- and control-antibody treated rats.

Since SLAMF7 is an inhibitory receptor in human monocytes and T cells, SLAMF7 activation prevents proinflammatory chemokine responses, and thus, prevents T cell activation and recruitment to the islets. These results establish the development of a novel immunotherapy strategy that prevents T1D development. Prevention of T1D development in the spontaneously developing T1D BB rat provides a therapeutic strategy for preventing T1D development in high-risk population.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of decreasing the production of a pro-inflammatory cytokine in an activated cell of myeloid lineage of a subject in need thereof, the method comprising:
increasing at least one of signaling lymphocytic activation molecule F7 (SLAMF7) expression, SLAMF7 activity, and SLAMF7 signaling in the activated cell of myeloid lineage.

2. The method according to claim 1, wherein the pro-inflammatory cytokine is selected from the group consisting of interleukin-1 (IL-1P), interleukin-6 (IL-6), interleukin-17a (IL-17a), tumor necrosis factor α (TNFα), a chemokine, and combinations thereof.

3. The method according to claim 2, wherein the chemokine is C-X-C motif chemokine 9 (CXCL9), C-X-C motif chemokine 10 (CXCL10), C-X-C motif chemokine 11 (CXCL11), C-X-C motif chemokine 12 (CXCL12), or a combination thereof.

4. The method according to claim 1, wherein the subject in need thereof has a disorder selected from the group consisting of HIV-associated neurocognitive disorder (HAND), HIV-associated dementia (HAD), breast cancer, atherosclerosis, coronary artery disease, sepsis, systemic lupus erythematosus, myocardial infarction, Alzheimer's disease, Huntington's disease, multiple sclerosis, obesity, kidney disease, Rheumatoid arthritis, and a combination thereof.

5. The method according to claim 1, wherein increasing at least one of SLAMF7 expression, SLAMF7 activity, and SLAMF7 signaling comprises:
administering a safe and effective amount of nuclear factor-κβ (NF-κβ), palmitoyl cysteine serine lysine 4 (pamCSK4), histone deacetylase 1 activator (HDAC1), histone deacetylase 2 activator (HDAC2), p300 inhibitor, a CD3 activator, a CD28 activator, a DNA molecule capable of expressing a SLAMF7 gene or gene fraction, a component that decreases YY1 levels, a component that prevents or decreases acetylation of YY1, interferon α (IFNα), interferon β (IFNβ), interferon γ (IFNγ), lipopolysaccharide (LPS), Adenovirus, polyinosinic:polycytidylic acid (poly I:C), flagelin, guanosine-adenosine 2′,3′-cyclic monophosphate (2′3′-cGAMP), resiquimod (R848), SLAMF7 agonistic antibodies, SLAMF7 agonistic antibody fractions, a hybrid molecule comprising a portion of SLAMF7 and an antibody Fc fragment, a small molecule agonist of SLAMF7, a peptide, a micelle or liposome having recombinant SLAMF7 on a surface, or a combination thereof to the subject.

6. The method according to claim 5, wherein the DNA molecule is a plasmid or a minicircle.

7. The method according to claim 5, wherein administering the DNA molecule comprises transfecting cells of the myeloid lineage by a virus, electroporation, direct microinjection, laser-mediated transfection, cationic lipid transfection, squeezing the cell to create an opening in the cell's membrane, or calcium phosphate transfection.

8. The method according to claim 1, wherein the cell of myeloid lineage is derived from a multipotential hematopoietic stem cell.

9. The method according to claim 8, wherein the cell of myeloid lineage is a monocyte, a neutrophil, or a microglia.

10. The method according to claim 1, wherein the cell of myeloid lineage is derived from an erythro-myeloid precursor in an embryonic yolk sac.

11. The method according to claim 10, wherein the cell of myeloid lineage is a microglia.

12. A method of decreasing the production of a pro-inflammatory cytokine in an activated cell of myeloid lineage of a subject in need thereof, the method comprising:
administering a safe and effective amount of a signaling lymphocytic activation molecule F7 (SLAMF7) agonist to the subject,
wherein the SLAMF7 agonist increases at least one of SLAMF7 expression, SLAMF7 activity, and SLAMF7 signaling in the activated cell of myeloid lineage, wherein the activated cell of myeloid lineage is a monocyte, neutrophil, or microglia that produces pro-inflammatory cytokines at a level that is higher than baseline.

13. The method according to claim 12, wherein the subject has HIV-associated neurocognitive disorder (HAND) or HIV-associated dementia (HAD).

14. The method according to claim 13, further comprising:
treating the subject with combination antiretroviral therapy (cART).

15. The method according to claim 12, wherein the SLAMF7 agonist is a SLAMF7 agonistic antibody, a SLAMF7 agonistic antibody fragment, a hybrid molecule comprising a portion of SLAMF7 and an antibody Fc fragment, a small molecule, a peptide, or an agent that increases SLAMF7 expression and signaling in the activated cell of myeloid lineage.

16. A method of inhibiting human immunodeficiency virus (HIV) from infecting a cell of either myeloid lineage or lymphoid lineage, the method comprising:
modulating the cell to at least one of decrease an amount of chemokine receptor type 5 (CCR5) in a cell membrane of the cell or increase an amount of C-C motif chemokine ligand 3 like 1 (CCL3L1) expressed in the cell.

17. The method according to claim 16, wherein the modulating is performed by increasing at least one of signaling lymphocytic activation molecule F7 (SLAMF7) expression, SLAMF7 activity, and SLAMF7 signaling in the cell.

18. The method according to claim 17, wherein the increasing at least one of SLAMF7 expression, SLAMF7 activity, and SLAMF7 signaling in the cell is performed by contacting the cell with palmitoyl cysteine serine lysine 4 (pamCSK4), histone deacetylase 1 activator (HDAC1), histone deacetylase 2 activator (HDAC2), a p300 inhibitor, a CD3 activator, a CD28 activator, a DNA molecule capable of expressing a SLAMF7 gene, a component that decreases YY1 levels, a component that prevents or decreases acetylation of YY1, interferon α (IFNα), interferon β (IFNβ), interferon γ (IFNγ), lipopolysaccharide (LPS), Adenovirus, polyinosinic:polycytidylic acid (poly I:C), flagelin, guanosine-adenosine 2′,3′-cyclic monophosphate (2′3′-cGAMP), resiquimod (R848), SLAMF7 agonistic antibodies, SLAMF7 agonistic antibody fractions, a small molecule agonist of SLAMF7, a peptide, a micelle or liposome having recombinant SLAMF7 on a surface, or a combination thereof.

19. The method according to claim 16, wherein the cell is a monocyte or a T cell.

20. The method according to claim 16, wherein the cell is in a subject having human immunodeficiency virus (HIV) or in a subject at risk of having HIV.
